SEQUENCE LISTING (2 sequences)

SEQ ID NO: 1 - protein, 23 aa, Unknown
  MAMMALIAN
KAFAKLAARLYRKALARQLGVAA

SEQ ID NO: 2 - protein, 21 aa, Unknown
  MAMMALIAN
FAKLAARLYRKALARQLGVAA

(19) United States
(12) Patent Application Publication
(10) Pub. No.: US 2009/0269406 A1
(43) Pub. Date: Oct. 29, 2009

(54) Therapeutic uses of biocompatible biogel compositions

(76) Inventors: Alyssa Panitch, W. Lafayette, IN (US); Brandon Seal, W. Lafayette, IN (US)

Correspondence Address: GREENBERG TRAURIG, LLP, 200 PARK AVE., P.O. BOX 677, FLORHAM PARK, NJ, 07932, US

(21) Appl. No.: 12/384,421

(22) Filed: Apr. 2, 2009

Related U.S. Application Data
(60) Provisional application No. 61/041,705, filed on Apr. 2, 2008.

(51) Int. Cl. A61K 9/10; A61K 35/00; A61K 38/02; A61K 39/395; A61K 38/16; A61K 35/12

(52) U.S. Cl. 424/487; 424/484; 424/93.1; 514/2; 424/130.1; 514/13; 424/93.7

(57) ABSTRACT

The present invention relates to biocompatible biogel compositions and methods of drug delivery. The biocompatible biogel is a physical polymer matrix formed via affinity interactions between its components. The components of the biocompatible biogel comprise a cationic component, an anionic component, and optionally a therapeutic agent.

### CROSS-REFERENCE TO RELATED APPLICATIONS

This application claims benefit of priority to U.S. Patent Application Ser. No. 61/041,705, filed Apr. 2, 2008, which is incorporated herein by reference in its entirety.

### FIELD OF THE INVENTION

The present invention relates to the fields of polymer chemistry, tissue engineering scaffolds, and methods for drug delivery.

### BACKGROUND OF THE INVENTION

Polymeric-based systems for in vivo delivery of therapeutic agents are the subject of active study. Current polymeric-based systems generally suffer from one or more drawbacks. First, many covalent polymeric networks require implantation since most cannot be delivered in situ. Second, those covalent polymeric networks that can be formed in situ require time for chemical, light or enzymatic initiation. The time for polymerization may be relatively brief (i.e., several seconds to a few minutes), however, any time spent during polymerization allows the components of a delivery system to diffuse away. Third, chemical and photo-initiators often are toxic, while enzymatic initiators depend on enzyme kinetics. Fourth, covalent delivery vehicles cannot degrade without the incorporation of hydrolytic or enzymatic degradation sites. The degradation of networks incorporating chemistries for hydrolytic degradation is nonspecific and can be difficult to control. Further, the degradation of networks incorporating chemistries for enzymatic recognition and cleavage depends on, among other things, enzyme diffusion into and through the network and local regulation of enzyme expression.

The formation of physical polymeric systems can involve time (sometimes several hours), temperatures, pH and salt concentrations that are outside the range of physiological conditions. Similarly, delivery systems formed via covalent cross-linking of a polymeric material by polysaccharide-binding polypeptides do not immediately form networks. These covalent gels cannot be reformed if the covalent chemical bonds are broken, and cannot change their shape within a dynamic, remodeling environment, such as those that exist within normal, healing or regenerating tissues.

It would seem to a properly informed artisan that an ideal physical system based on the biological affinity of the components would form a gel-like material immediately at a physiologically relevant (i) temperature, (ii) pH, and (iii) salt concentration. Such an ideal physical system would be capable of reforming after a mechanical or environmental insult or perturbation, and also would be capable of modifying its shape to accommodate alterations in in vivo geometry or surroundings. Since such physical systems would assemble in a manner that mimics assembly of the extracellular matrix (physical gelation), they are more appropriate for in vivo use than are covalently crosslinked gels. Therapeutics with affinity for a component of such physical system could be sequestered within the system and released based on the relative affinity between the therapeutic and the system.

There is a need for nontoxic delivery systems based on biological affinity, and methods for drug delivery using such systems remains unmet.

### SUMMARY OF THE INVENTION

According to one aspect, the present invention provides a method for treating a wound with a biocompatible biogel composition, the method comprising the steps: (a) providing a biocompatible biogel composition comprising (i) a cationic component, wherein the cationic component comprises a hydrophilic polymer having a molecular weight greater than about 3000 g/mole, but less than about 10,000,000 g/mole, to which at least about 3 cationic oligomers, but no more than 1,000,000 cationic oligomers is grafted; and (ii) an anionic component; and (iii) optionally a therapeutically effective amount of a therapeutic agent; and (b) forming a biocompatible matrix to support wound healing. According to one embodiment, the therapeutic agent is selected from the group consisting of an analgesic agent, a biological agent, a pharmaceutical composition, a growth factor, a cell or a polypeptide. According to some such embodiments, the therapeutic agent is a microparticle form. According to some such embodiments, the therapeutic agent is a nanoparticle form. According to some such embodiments, the biological agent is an isolated cell. According to some such embodiments, the biological agent is an isolated peptide, and isolated polypeptide, an isolated antibody or an isolated active portion, fragment or derivative thereof. According to some such embodiments, the biological agent is an isolated polypeptide having an amino acid sequence according to general formula I: Z1-X1-X2-X3-X4-X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid. According to some such embodiments, the biological agent is an isolated polypeptide having an amino acid sequence according to general formula I: Z1-X1-X2-X3-X4 X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent. According to some such embodiments, X4 is R, X5 is Q and X8 is V. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion. According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the hydrophilic polymer comprises acrylamide, styrene, acrylic acid and a polymerization initiator. According to some such embodiments, the hydrophilic polymer comprises (i) 50% acrylamide, (ii) 15% styrene, (iii) 35% acrylic acid and (iv) 2,2′-azobisisobutyronitrile 1/100 molar ratio to monomers. According to some such embodiments, the hydrophilic polymer comprises (i) 50% acrylamide, (ii) 15% styrene, (iii) 35% acrylic acid and (iv) 2,2′-azobisisobutyronitrile 1/200 molar ratio to monomers. According to some such embodiments, the acrylic acid is functionalized with a guanidyl group. According to some such embodiments, the guanidyl group is agmatine sulfate. According to some such embodiments, the guanidyl group is of arginine, or a derivative thereof. According to some such embodiments, According to some such embodiments, the wound is a nonhealing wound. According to some such embodiments, the nonhealing wound is a venous ulcer. According to some such embodiments, the nonhealing wound is a diabetic ulcer. According to some such embodiments, the nonhealing wound is a nonhealing burn. According to some such embodiments, the wound is a neural wound.

According to another aspect, the present invention provides a method for supporting differentiation of isolated differentiable cells into a mature phenotype, the method comprising steps: (1) providing a biocompatible biogel composition comprising: (a) a biogel for growing isolated differentiable cells, the biogel comprising (i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and (ii) an anionic component; and (b) isolated differentiable cells; (2) administering the biocompatible biogel composition into a region of interest to a subject in need thereof; (3) forming a tissue scaffold to support differentiation of isolated cells into a mature phenotype. According to one embodiment, the method according to claim 23, wherein the isolated differentiable cells are multipotent human mesenchymal cells. According to some such embodiments, the biogel supports differentiation of the isolated differentiable cells into a mature phenotype, and wherein the mature phenotype is a chondrocyte. According to some such embodiments, the biogel supports differentiation of the isolated differentiable cells into a mature phenotype, wherein the mature phenotype is a myocyte. According to some such embodiments, the biogel supports differentiation of the isolated differentiable cells into a mature phenotype, wherein the mature phenotype is an osteoblast. According to some such embodiments, the region of interest is in or adjacent to a bone tissue. According to some such embodiments, the region of interest is in or adjacent to a cardiac tissue. According to some such embodiments, the region of interest is in or adjacent to a neural tissue. According to some such embodiments, the region of interest is in or adjacent to a wound. According to some such embodiments, the region of interest is in or adjacent to a nonhealing wound. According to some such embodiments, the hydrophilic polymer comprises acrylamide, styrene, acrylic acid and a polymerization initiator. According to some such embodiments, the hydrophilic polymer comprises (i) 50% acrylamide, (ii) 15% styrene, (iii) 35% acrylic acid and (iv) 2,2′-azobisisobutyronitrile 1/100 molar ratio to monomers. According to some such embodiments, the hydrophilic polymer comprises (i) 50% acrylamide, (ii) 15% styrene, (iii) 35% acrylic acid and (iv) 2,2′-azobisisobutyronitrile 1/200 molar ratio to monomers. According to some such embodiments, wherein the acrylic acid is functionalized with a guanidyl group. According to some such embodiments, the guanidyl group is agmatine sulfate. According to some such embodiments, the guanidyl group is of arginine, or a derivative thereof.

According to another aspect, the present invention provides a biomedical device comprising a biocompatible biogel composition disposed on the device, the biogel composition comprising (i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and (ii) an anionic component; and wherein the biogel composition improves at least one anti-adhesive property of the device. According to one embodiment, the biocompatible biogel composition further comprises a therapeutic agent. According to some such embodiments, the therapeutic agent is a microparticle form. According to some such embodiments, the therapeutic agent is a nanoparticle form. According to some such embodiments, the therapeutic agent is selected from the group consisting of an analgesic agent, an antimicrobial agent, a steroid agent, a chemotherapeutic agent, a biological agent, a pharmaceutical composition, a growth factor, a cell, or a polypeptide. According to some such embodiments, the biological agent is an isolated cell. According to some such embodiments, the biological agent is an isolated peptide, an isolated polypeptide, an isolated antibody or an isolated active portion, a fragment, or a derivative thereof. According to some such embodiments, the biological agent is an isolated polypeptide having an amino acid sequence according to general formula I: Z1-X1-X2-X3-X4 X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid. According to some such embodiments, the biological agent is an isolated polypeptide having an amino acid sequence according to general formula I: Z1-X1-X2-X3-X4 X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent. According to some such embodiments, X4 is R, X5 is Q and X8 is V. According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion. According to some such embodiments, the hydrophilic polymer comprises acrylamide, styrene, acrylic acid and a polymerization initiator. According to some such embodiments, the hydrophilic polymer comprises (i) 50% acrylamide, (ii) 15% styrene, (iii) 35% acrylic acid and (iv) 2,2′-azobisisobutyronitrile 1/100 molar ratio to monomers. According to some such embodiments, the hydrophilic polymer comprises (i) 50% acrylamide, (ii) 15% styrene, (iii) 35% acrylic acid and (iv) 2,2′-azobisisobutyronitrile 1/200 molar ratio to monomers. According to some such embodiments, the acrylic acid is functionalized with a guanidyl group. According to some such embodiments, the guanidyl group is agmatine sulfate. According to some such embodiments, the guanidyl group is of arginine, or a derivative thereof.

According to another aspect, the present invention provides a method for treating inflammation with a biocompatible biogel composition, the method comprising the steps: (i) providing a biocompatible biogel composition comprising (a) a cationic component; wherein the cationic component comprises a hydrophilic polymer having a molecular weight great than about 3000 g/mole, but less than about 10,000,000 g/mole, to which at least about 3, but no more than 1,000,000 cationic oligomers is grafted; (b) an anionic component; and (c) a therapeutically effective amount of a therapeutic agent; (ii) administering the biocompatible biogel composition of step (i) to a region of interest within a subject in need thereof, wherein the region of interest contains or is adjacent to an area of inflammation; thereby reducing the inflammation. According to one embodiment, the therapeutic agent is selected from the group consisting of an analgesic agent, an antimicrobial agent, a steroid agent, a chemotherapeutic agent, a biological agent, a pharmaceutical composition, a growth factor, a cell, or a polypeptide. According to some such embodiments, the therapeutic agent is a microparticle form. According to some such embodiments, the therapeutic agent is a nanoparticle form. According to some such embodiments, the biological agent is an isolated cell. According to some such embodiments, the biological agent is an isolated peptide, an isolated polypeptide, an isolated antibody or an isolated active portion, fragment or derivative thereof. According to some such embodiments, the biological agent is an isolated polypeptide having an amino acid sequence according to general formula I: Z1-X1-X2-X3-X4 X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid. According to some such embodiments, the biological agent is an isolated polypeptide having an amino acid sequence according to general formula I: Z1-X1-X2-X3-X4 X5-X6-X7-X8-X9-X10-Z2 wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, T, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent. According to some such embodiments, X4 is R, X5 is Q and X8 is V. According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion. According to some such embodiments, the hydrophilic polymer comprises acrylamide, styrene, acrylic acid and a polymerization initiator. According to some such embodiments, the hydrophilic polymer comprises (i) 50% acrylamide, (ii) 15% styrene, (iii) 35% acrylic acid and (iv) 2,2′-azobisisobutyronitrile 1/100 molar ratio to monomers. According to some such embodiments, the hydrophilic polymer comprises (i) 50% acrylamide, (ii) 15% styrene, (iii) 35% acrylic acid and (iv) 2,2′-azobisisobutyronitrile 1/200 molar ratio to monomers. According to some such embodiments, the acrylic acid is functionalized with a guanidyl group. According to some such embodiments, the guanidyl group is agmatine sulfate. According to some such embodiments, the guanidyl group is of arginine, or a derivative thereof. According to some such embodiments, the inflammatory disorder is selected from the group consisting of hyperplastic scarring, keloids, rheumatoid arthritis, chronic obstructive pulmonary disease, atherosclerosis, intimal hyperplasia, Crohn's disease, inflammatory bowel disease, osteoarthritis, Lupus, tendonitis, psoriasis, gliosis, inflammation, type II diabetes mellitus, type I diabetes mellitus, Alzheimer's disease, and an adhesion. According to some such embodiments, the inflammatory disorder comprises glial scarring.

According to another aspect, the present invention provides a tissue filler to fill a tissue void, comprising (a) a gel-like system comprising (i) a cationic component, wherein the cationic component comprises a hydrophilic polymer having a molecular weight great than about 3000 g/mole, but less than about 10,000,000 g/mole, to which at least about 3, but no more than 1,000,000 cationic oligomers is grafted; and (ii) an anionic component; (b) and optionally a therapeutically effective amount of a therapeutic agent.

### BRIEF DESCRIPTION OF THE DRAWINGS

FIG. 1 shows a plot of viable cells (%) versus peptide concentration (μM). 3T3 fibroblasts were incubated with various doses of W-PBD1 () and dG-PBD1 (▪) peptide (n=3). Viability was measured using a colorimetric metabolic assay based on the reduction of a tetrazolium salt into a formazan product. PBD refers to polysaccharide binding domain (PBD).

### DETAILED DESCRIPTION OF THE INVENTION

### I. Biocompatible Biogel Compositions

The present invention provides a biocompatible biogel composition to carry therapeutic agents, including, but not limited to, pharmacological agents, cells, microparticles, and nanoparticles, pharmaceutical compositions, and polypeptides for delivery into regions of interest within a subject. The biocompatible biogel composition comprises a biocompatible biogel. The biocompatible biogel components form a gel or gel-like material upon mixture at physiologically relevant temperature, pH and salt concentrations that is capable of reforming after a mechanical or environmental insult or perturbation, and is capable of modifying its shape to accommodate alterations in in vivo geometry or surroundings. Thus, the biocompatible biogel composition assembles in a manner that mimics assembly of the extracellular matrix (physical gelation), and therefore has unique properties relative to covalently crosslinked gels. Many of these unique properties are maintained even when the biocompatible biogel composition is crosslinked following initial system assembly.

According to one aspect, the present invention provides a biocompatible biogel composition comprising: (a) a biocompatible biogel comprising: (i) a cationic component; (ii) an anionic component; and (b) a therapeutically effective amount of a therapeutic agent.

### 1. Biocompatible Biogel

According to another aspect, the present invention provides a biocompatible biogel comprising: (i) a cationic component and (ii) an anionic component. According to one embodiment, the biocompatible biogel further comprises a therapeutic agent.

The term “biocompatible” as used herein refers to causing no clinically relevant tissue irritation or necrosis at a local site necessitating removal of a device prior to the end of therapy based on a clinical risk/benefit assessment. The term “therapy” as used herein refers to the treatment of a disease or disorder, as by some remedial, rehabilitating or curative process. The phrase “end of therapy” as used herein means the cessation of treatment of a disease or disorder.

The term “biogel” as used herein refers to a biocompatible gel or gel-like system that forms a physical polymer matrix based on affinity interactions between its components. According to the invention, a biogel contains a cationic component, an anionic component, and optionally, a therapeutic agent.

The term “gel” as used herein refers to a solid, jelly-like material that can have properties ranging from soft and weak to hard and tough. A gel is a substantially dilute crosslinked system, which exhibits limited or no flow when in the steady-state. By weight, gels may be mostly liquid, yet they behave like solids due to a three-dimensional crosslinked network within the liquid. It is the crosslinks within the fluid that give a gel its structure (hardness) and contribute to stickiness (tack).

The term “gel-like” as used herein refers to a substance or material resembling or having some or all the characteristics of a gel.

The term “subject” or “individual” or “patient” are used interchangeably to refer to a member of an animal species of mammalian origin, including but not limited to, a mouse, a rat, a cat, a goat, sheep, horse, hamster, ferret, pig, a dog, a platypus, a guinea pig, a rabbit and a primate, such as, for example, a monkey, ape, or human.

### 2. Cationic Component

According to one embodiment, the cationic component of the biocompatible biogel of the invention comprises a hydrophilic polymer. According to another embodiment, the cationic component comprises a hydrophilic polymer having a molecular weight greater than about 3,000 g/mole, but less than about 10,000,000 g/mole, to which at least about 3 cationic oligomers, but no more than 1,000,000 cationic oligomers, are grafted. According to another embodiment, the cationic component comprises a hydrophilic polymer of a molecular weight from about 3,000 g/mole to about 5,000,000 g/mole, to which at least 3 cationic oligomers are grafted, but to which no more than 1,000,000 cationic oligomers are grafted. According to another embodiment, the cationic component comprises a hydrophilic polymer of a molecular weight from about 3,000 g/mole to about 2,500,000 g/mole, to which at least 3 cationic oligomers are grafted, but to which no more than 1,000,000 cationic oligomers are grafted. According to another embodiment, the cationic component comprises a hydrophilic polymer of a molecular weight from about 3,000 g/mole to about 1,000,000 g/mole, to which at least 3 cationic oligomers are grafted, but to which no more than 1,000,000 cationic oligomers are grafted. According to another embodiment, the cationic component comprises a hydrophilic polymer of a molecular weight from about 3,000 g/mole to about 500,000 g/mole, to which at least 3 cationic oligomers are grafted, but to which no more than 1,000,000 cationic oligomers are grafted. According to another embodiment, the cationic component comprises a hydrophilic polymer of a molecular weight from about 3,000 g/mole to about 100,000 g/mole, to which at least 3 cationic oligomers are grafted, but to which no more than 1,000,000 cationic oligomers are grafted.

The term “hydrophilic” refers to substances having a strong affinity for water.

The term “oligomer” as used herein refers to a polymer molecule consisting of a small number (about 1 to about 300) of monomers.

The term “polymer” as used herein refers to a macromolecular substance composed of one or more repeating atomic groups, called monomers, and includes linear, branched, and cross-linked polymers, and combinations thereof. The polymer can comprise copolymers, block copolymers, graft copolymers, alternating copolymers, and random copolymers.

The term “copolymer” as used herein refers to a polymer composed of two or more different monomer units. Biological copolymers include, but are not limited to, proteins, polysaccharides, DNA, and RNA. Synthetic copolymers include, but are not limited to, poly(lactic acid-co-glycolic acid).

The term “block copolymer” as used herein refers to a polymer composed of linear segments containing one or more monomers of the same type, which are covalently attached to at least one other segment containing one or more monomers of a different type. Block copolymers include, but are not limited to, copolymers of ethylene glycol and propylene glycol.

The term “graft copolymer” as used herein refers to one or more polymer chain to which are covalently attached, along their backbone, one or more linear or branched chains containing one or more monomer unit.

The term “alternating copolymer” as used herein refers to polymer chains containing either alternating monomers of a different type or alternating blocks of monomers of different type.

The term “random copolymer” as used herein refers to two or more monomer units that do not occur along the backbone in an alternating fashion. Random copolymers include, but are not limited to, poly(acrylamide-co-N-isopropyl acrylamide).

The term “branched polymer” as used herein refers to a non-linear arrangement of monomers. Branched polymers include, but are not limited to, polyethylene glycol (PEG) star polymers, PEG comb polymers, and dendrimers.

The term “charged” polymer as used herein refers to both an inherently charged polymer and a polymer that becomes charged under specific environmental conditions (such as, for example, but not limited to, pH). Charged polymers include, but are not limited to, polymers with oxygen, hydroxyl, and carboxyl residues (such as poly(vinyl alcohol) polymethacrylate, polyfumerates, poly(n-isopropylacrylamide), and poly(vinyl pyrrolidone).

The term “ion” as used herein refers to an atom or radical that has lost or gained one or more electrons and has thus acquired an electric charge. The term “cation” as used herein refers to an ion having a positive charge. The term “anion” as used herein refers to an ion having a negative charge.

Hydrophilic polymers include, but are not limited to, poly(ethylene glycol), poly(ethylene oxide), poly(vinyl alcohol), poly(acrylic acid), poly(ethylene-co-vinyl alcohol), poly(vinyl pyrrolidone), poly(ethyloxazoline), poly(ethylene oxide)-co-poly(propylene oxide) copolymers, polyacrylates, polymethacrylates, poly(hydroxyethyl methacrylate), polyfumarates, poly(n-isopropylacrylamide), dextran, hyaluronic acid, and elastomeric polypeptides or derivatives thereof. According to another embodiment, the hydrophilic polymer is dextran to which greater than about 3 poly(vinyl amine) oligomers (having a molecular weight of from about 500-5000 daltons) have been grafted.

According to another embodiment, the cationic oligomers are synthetic polymers that are grafted on a hydrophilic polymer. Examples of synthetic polymers include, but are not limited to, poly(vinyl amine), poly(allyl amine), poly(acrylate amines), synthetic heparin binding peptide mimics [Choi S, Clements D J, Pophristic V, Ivanov I, Vemparala S, Bennett J S, Klein M L, Winkler J D, DeGrado W F, The design and evaluation of heparin-binding foldamers, Angewandte Chemie International Edition, 2005, 44(41): 6685-6689], synthetic antimicrobial peptide mimics [US Patent 20060024264; Liu D, DeGrado W F, De novo design, synthesis, and characterization of antimicrobial beta-peptides, J Am Chem Soc, 2001, 123(31): 7553-7559], urea oliogomers [Violette A, Fournel S, Lamour K, Chaloin O, Frisch B, Briand J, Monteil H, Guichard G, Mimicking helical antibacterial peptides with nonpeptidic folding oligomers, Chemistry and Biology, 2006, 13(5): 531-538; Tang H, Doerksen R J, Tew G N, Synthesis of urea oligomers and their antibacterial activity, Chem Commun, 2005, 1537-1539; Tew G N, Liu D, Chen B, Doerksen R J, Kaplan J, Carroll P J, Klein, M L, DeGrado W F, De novo design of biomimetic antimicrobial polymers, PNAS, 2002, 99(8): 5110-5114], poly(vinyl formamide), poly(N,N-diethylamino ethyl methacrylate), poly(N,N-dimethylamino ethyl acrylate), poly(diethylamino ethylstyrene), poly(N,N-diethylamino ethyl methacrylate), poly(N,N-diethylamino ethyl acrylate), poly(t-butylamino ethyl methacrylate), poly(t-butylamino ethyl acrylate), poly(aminoethyl methacrylate), poly(aminoethyl acrylate), poly(diisopropylaminoethyl methacrylate), poly(diisopropylaminoethyl acrylate), poly(N-morpholinoethyl acrylate), poly(N-morpholinoethyl methacrylate), poly(dimethylaminoeopentyl acrylate), poly(dimethylaminoeopentyl methacrylate), poly(diallylamine), poly(diallyldimethylammonium), poly(methacryloyl lysine), poly(N-2-aminoethyl methacrylamide), poly(N-3-aminopropyl methacrylamide), poly(N-t-BOC-aminopropyl methacrylamide), poly(N-2-N,N-dimethylamino ethyl methacrylamide), poly(N-3-N,N-dimethylamino propyl acrylamide), poly(N-3-N,N-dimethylamino propyl methacrylamide, poly(4-aminobutyl guanidine), and polymers with pendant primary amines, secondary amines, tertiary amines, and/or guanidinyl groups.

The term “PB polypeptides” as used herein refers to polysaccharide-binding polypeptides. The term “binding” as used herein refers to combine with or to form a bond with. The bond formed may be, but is not limited to, a chemical bond, or a physical bond. For example, the binding of polysaccharide-binding polypeptides refers to the ability to form physical bonds.

The hydrophilic polymers may be modified with one or more blocks comprising one or more degradable moieties polymerized on one or both ends of the polymer to confer additional degradability. As a non-limiting example, poly(ethylene glycol) is not inherently degradable; however, the ends of the poly(ethylene glycol) chains may be modified with degradable polyesters. Polysaccharide binding (PB) polypeptides may be covalently bound to the degradable polyesters. Once the degradable polyester degrades, the PB polypeptides are released from the polymer, and the composition formed through interaction of the PB polypeptides and the negatively charged polysaccharides falls apart.

Alternatively, the hydrophilic polymer may be polymerized with degradable oligomers to form degradable block copolymers, which may serve to increase the loss of coordination of the composition, leading to its degradation. As a non-limiting example, the block copolymers described above may be made with non-degradable polymers and a degradable block, for example, poly(ethylene glycol)-degradable block-poly(propylene glycol). These blocks may be composed of, for example, but not limited to, lactic acid, glycolic acid, ε-caprolactone, lactic-co-glycolic acid oligomers, trimethylene carbonate, anhydrides, and amino acids. This list is not exhaustive; other oligomers also may be used for block copolymers. The blocks do not have to be hydrophilic, as long as the overall polymer remains hydrophilic.

As the molecular weight of the hydrophilic polymer increases, the viscosity of the composition naturally will increase. An increase in the viscosity, however, does not imply that the composition only may be in the form of a viscous solution. If a hydrophilic polymer has relatively few sites to which cationic oligomers may covalently bind, then compositions with larger molecular weight polymers would have a decreased “crosslink” density. As a result, such compositions are more likely to be in the form of a viscous solution. As a non-limiting example, a 4-arm polyethylene glycol (PEG) (avg. MW 10,000 g/mol) covalently attached to four PB polypeptides may form a physical gel when mixed with heparin or dextran sulfate. A similar composition comprising 4-arm PEG with an average molecular weight of 100,000 g/mol covalently attached to four PB polypeptides would have a lower cross link density. The latter composition may assume the form of a viscous solution. Polymers such as, for example, dextran contain many potential sites to which a polypeptide may be coupled. The number of binding sites may scale with size for any polymer composed of monomers with free functional groups. For example, dextran may be modified such that there are three sites per monomer to which a cationic oligomer, such as, for example, but not limited to, a polypeptide, could be covalently bound. As a result, a dextran molecule with an average molecular weight of 70,000 g/mol may have several hundred cationic oligomers covalently bound to it. As a result, the crosslink density is very high, which is likely to result in the formation of a physical gel when mixed with a negatively charged polysaccharide.

According to some embodiments, the hydrophilic polymer may comprise polypeptide subunits. The polypeptide may be a naturally occurring, chemically synthesized, or recombinant polypeptide. Polypeptides may be especially useful where additional degradability of the composition is desired, since the polypeptide portion of the polymer will be subject to proteolysis. In addition, such polypeptides may be selected or engineered to include other desirable features for a given application, including, but not limited to, binding sites for cells or other proteins. Polypeptides for use in the polymer of the present invention include, but are not limited to, collagens, laminins, fibronectin, albumin, and vitronectin. According to some such embodiments, a protein polymer does not comprise fibrin.

Where the hydrophilic polymer of the present invention comprises polypeptide subunits, the cationic oligomers, such as for example, but not limited to, PB polypeptides, may be covalently bound to the polypeptide, via, for example, the functional groups on cysteine, tyrosine, and/or lysine residues. Alternatively, when the polymer of the present invention comprises polypeptide subunits, the polypeptide subunit may be selected or engineered to include one or more PB polypeptides within the polypeptide sequence. Such embodiments may permit tighter control of the relative ratio of polymer to PB polypeptide to negatively charged polysaccharide compared to the embodiments described above.

According to some embodiments, the composition further comprises cationic PB polypeptides and negatively charged polysaccharides, both of which are hydrophilic. A hydrophilic polymer allows the PB polypeptides to associate more freely with the polysaccharides, and provides an increased degree of hydration of the composition, which leads to a more homogenous composition (i.e., less water is excluded).

According to some embodiments, the hydrophilic polymer has a degree of polymerization from about 20 to about 200. According to some embodiments, the hydrophilic polymer has a degree of polymerization from about 20 to about 150. According to some embodiments, the hydrophilic polymer has a degree of polymerization from about 20 to about 100. According to some embodiments, the hydrophilic polymer has a degree of polymerization from about 20 to about 50. The phrase “degree of polymerization” as used herein refers to the number of monomeric units in a macromolecule or oligomer molecule, a block or chain. The term “monomer” as used herein refers to a small molecule that may become chemically bonded to other monomers to form a polymer.

According to some embodiments, the hydrophilic polymer comprises from 0% aromatic containing monomer to about 20% aromatic containing monomer. According to some embodiments, the hydrophilic polymer contains about 1% aromatic containing monomer. According to some embodiments, the hydrophilic polymer contains about 2% aromatic containing monomer. According to some embodiments, the hydrophilic polymer contains about 3% aromatic containing monomer. According to some embodiments, the hydrophilic polymer contains about 4% aromatic containing monomer. According to some embodiments, the hydrophilic polymer contains about 5% aromatic containing monomer. According to some embodiments, the hydrophilic polymer contains about 6% aromatic containing monomer. According to some embodiments, the hydrophilic polymer contains about 7% aromatic containing monomer. According to some embodiments, the hydrophilic polymer contains about 8% aromatic containing monomer. According to some embodiments, the hydrophilic polymer contains about 9% aromatic containing monomer. According to some embodiments, the hydrophilic polymer contains about 10% aromatic containing monomer. According to some embodiments, the hydrophilic polymer contains about 11% aromatic containing monomer. According to some embodiments, the hydrophilic polymer contains about 12% aromatic containing monomer. According to some embodiments, the hydrophilic polymer contains about 13% aromatic containing monomer. According to some embodiments, the hydrophilic polymer contains about 14% aromatic containing monomer. According to some embodiments, the hydrophilic polymer contains about 15% aromatic containing monomer. According to some embodiments, the hydrophilic polymer contains about 16% aromatic containing monomer. According to some embodiments, the hydrophilic polymer contains about 17% aromatic containing monomer. According to some embodiments, the hydrophilic polymer contains about 18% aromatic containing monomer. According to some embodiments, the hydrophilic polymer contains about 19% aromatic containing monomer. According to some embodiments, the hydrophilic polymer contains about 20% aromatic containing monomer.

According to some embodiments, the hydrophilic polymer comprises about 0% phenol containing monomer to about 20% phenol containing monomer. According to some embodiments, the hydrophilic polymer contains about 1% phenol containing monomer. According to some embodiments, the hydrophilic polymer contains about 2% phenol containing monomer. According to some embodiments, the hydrophilic polymer contains about 3% phenol containing monomer. According to some embodiments, the hydrophilic polymer contains about 4% phenol containing monomer. According to some embodiments, the hydrophilic polymer contains about 5% phenol containing monomer. According to some embodiments, the hydrophilic polymer contains about 6% phenol containing monomer. According to some embodiments, the hydrophilic polymer contains about 7% phenol containing monomer. According to some embodiments, the hydrophilic polymer contains about 8% phenol containing monomer. According to some embodiments, the hydrophilic polymer contains about 9% phenol containing monomer. According to some embodiments, the hydrophilic polymer contains about 10% phenol containing monomer. According to some embodiments, the hydrophilic polymer contains about 11% phenol containing monomer. According to some embodiments, the hydrophilic polymer contains about 12% phenol containing monomer. According to some embodiments, the hydrophilic polymer contains about 13% phenol containing monomer. According to some embodiments, the hydrophilic polymer contains about 14% phenol containing monomer. According to some embodiments, the hydrophilic polymer contains about 15% phenol containing monomer. According to some embodiments, the hydrophilic polymer contains about 16% phenol containing monomer. According to some embodiments, the hydrophilic polymer contains about 17% phenol containing monomer. According to some embodiments, the hydrophilic polymer contains about 18% phenol containing monomer. According to some embodiments, the hydrophilic polymer contains about 19% phenol containing monomer. According to some embodiments, the hydrophilic polymer contains about 20% phenol containing monomer.

According to some embodiments, the hydrophilic polymer comprises about 0% benzyl containing monomer to about 20% benzyl containing monomer. According to some embodiments, the hydrophilic polymer contains about 1% benzyl containing monomer. According to some embodiments, the hydrophilic polymer contains about 2% benzyl containing monomer. According to some embodiments, the hydrophilic polymer contains about 3% benzyl containing monomer. According to some embodiments, the hydrophilic polymer contains about 4% benzyl containing monomer. According to some embodiments, the hydrophilic polymer contains about 5% benzyl containing monomer. According to some embodiments, the hydrophilic polymer contains about 6% benzyl containing monomer. According to some embodiments, the hydrophilic polymer contains about 7% benzyl containing monomer. According to some embodiments, the hydrophilic polymer contains about 8% benzyl containing monomer. According to some embodiments, the hydrophilic polymer contains about 9% benzyl containing monomer. According to some embodiments, the hydrophilic polymer contains about 10% benzyl containing monomer. According to some embodiments, the hydrophilic polymer contains about 11% benzyl containing monomer. According to some embodiments, the hydrophilic polymer contains about 12% benzyl containing monomer. According to some embodiments, the hydrophilic polymer contains about 13% benzyl containing monomer. According to some embodiments, the hydrophilic polymer contains about 14% benzyl containing monomer. According to some embodiments, the hydrophilic polymer contains about 15% benzyl containing monomer. According to some embodiments, the hydrophilic polymer contains about 16% benzyl containing monomer. According to some embodiments, the hydrophilic polymer contains about 17% benzyl containing monomer. According to some embodiments, the hydrophilic polymer contains about 18% benzyl containing monomer. According to some embodiments, the hydrophilic polymer contains about 19% benzyl containing monomer. According to some embodiments, the hydrophilic polymer contains about 20% benzyl containing monomer.

According to some embodiments, the hydrophilic polymer comprises about 0% stryene to about 20% styrene.

According to another embodiment, the hydrophilic polymer comprises about 20% guanidino containing monomer to about 100% guanidino containing monomer. According to some such embodiments, the hydrophilic polymer comprises about 0% small polar or nonpolar pendant group containing monomer. According to some such embodiments, the small polar or nonpolar pendant group is that of acrylamide. According to some embodiments, the hydrophilic polymer comprises about 20% guanidino containing monomer and about 80% small polar or nonpolar pendant group containing monomer. According to some embodiments, the hydrophilic polymer comprises about 30% guanidino containing monomer and about 70% small polar or nonpolar pendant group containing monomer. According to some embodiments, the hydrophilic polymer comprises about 40% guanidino containing monomer and about 60% small polar or nonpolar pendant group containing monomer. According to some embodiments, the hydrophilic polymer comprises about 50% guanidino containing monomer and about 50% small polar or nonpolar pendant group containing monomer. According to some embodiments, the hydrophilic polymer comprises about 60% guanidino containing monomer and about 40% small polar or nonpolar pendant group containing monomer. According to some embodiments, the hydrophilic polymer comprises about 70% guanidino containing monomer and about 30% small polar or nonpolar pendant group containing monomer. According to some embodiments, the hydrophilic polymer comprises about 80% guanidino containing monomer and about 20% small polar or nonpolar pendant group containing monomer. According to some embodiments, the hydrophilic polymer comprises about 90% guanidino containing monomer and about 10% small polar or nonpolar pendant group containing monomer. According to some embodiments, the hydrophilic polymer comprises about 100% guanidino containing monomer and about 0% small polar or nonpolar pendant group containing monomer.

According to some embodiments, the hydrophilic polymer comprises acrylamide, styrene, acrylic acid and a polymerization initiator. According to some embodiments, the polymer initiator is 2,2′-azobisisobutyronitrile (“AIBN”). According to some such embodiments, the hydrophilic polymer comprises about 50% acrylamide, about 15% styrene, about 35% acrylic acid and about a 1/100 molar ratio of AIBN to monomers. According to some such embodiments, the hydrophilic polymer comprises about 50% acrylamide, about 15% styrene, about 35% acrylic acid and about a 1/200 molar ratio of AIBN to monomers.

The term “functional group” as used herein refers to specific groups of atoms within molecules that are responsible for the characteristic chemical reactions of those molecules. Generally, the same functional group will undergo the same or similar chemical reaction(s) regardless of the size of the molecule it is a part of. However, its relative reactivity can be modified by nearby functional groups.

The term “functionalization” as used herein refers to the addition of functional groups onto the surface of a material by chemical synthesis methods. The functional group added may be subjected to ordinary synthesis methods to attach virtually any kind of organic compound onto the surface.

According to some embodiments, the hydrophilic polymer comprises guanidino groups on the backbone of the polymer. According to another embodiment, the hydrophilic polymer comprises an acrylic acid that is functionalized with N-hydroxsuccimide. According to some embodiments, the hydrophilic polymer comprises an acrylic acid that is functionalized with a guanidyl group. According to some such embodiments, the guanidyl group is agmatine sulfate. According to some such embodiments, the guanidyl group is of arginine, or a derivative thereof. According to another embodiment, the hydrophilic polymer comprises an acrylic acid that is functionalized with agmatine.

### 3. Anionic Component

According to another embodiment, the anionic component is an anionic polymer. According to some such embodiments, the anionic polymer is a polysaccharide. According to some such embodiments, the polysaccharide is a negatively charged polysaccharide.

The term “polysaccharide” refers to a carbohydrate containing more than three monosaccharide units per molecule linked glycosidically to each other in branched or unbranched chains, that is hydrolyzable to its monosaccharide subunits.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The negatively charged polysaccharides may consist of multiple molecules of a single type of polysaccharide, or may comprise more than one type of polysaccharide. The negatively charged polysaccharides may comprise polysaccharides that inherently are negatively charged. Examples of polysaccharides include, but are not limited to, heparin, heparan sulfate, chondroitin sulfate, keratan sulfate, dermatan sulfate, hyaluronic acid, dextran sulfate, alginate, fucan, lipopolysaccharide. According to another embodiment, the anionic polymer comprises a dextran sulfate having a molecular weight of about 7,000 g/mole to about 70,000 g/mole. The negatively charged polysaccharides may comprise polysaccharides that are derivatized to be negatively charged, including, but not limited to, dextran, dermatan and agarose that have been chemically modified to contain at least one negatively charged chemical moiety including, but not limited to, sulfate, phosphate and carboxylic groups.

The negatively charged polysaccharides may include, but are not limited to, sulfated polysaccharides, phosphorylated polysaccharides, and carboxylated polysaccharides.

According to another embodiment, dextran sulfate is the anionic component and copolymers of butylguanidinyl acrylamide and a vinyl or allyl monomer are grafted to dextran.

According to some embodiments, dextran sulfate is the anionic component and dextran-graft-vinyl amine is the cationic component.

The choice of the negatively charged polysaccharide may depend on the type of composition desired. Generally, the greater the negative charge of the negatively charged polysaccharide, the more ionic coordination among the components in the resulting composition. Thus, some negatively charged polysaccharides may not contain sufficient charge density to allow for the formation of a physical gel regardless of the concentration of the polysaccharide and the relative ratio between cationic polymer (i.e., hydrophilic polymer) and polysaccharide. These mixtures tend to form viscous solutions, especially when using higher molecular weight polysaccharides. Other negatively charged polysaccharides do contain sufficient charge density to allow for the formation of a physical gel.

### 4. Therapeutic Agent

According to another embodiment, the therapeutic agent is an analgesic agent, a steroid agent, a chemotherapeutic agent, a pharmaceutical composition or a biological agent.

The term “therapeutic agent” as used herein refers to a drug, molecule, nucleic acid, protein, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably herein.

The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest reduction or elimination of the progression of a disease manifestation. The term “therapeutically effective amount” or an “amount effective” of one or more of the active agents of the present invention is an amount that is sufficient to provide a therapeutic effect. Generally, an effective amount of the active agents that may be employed ranges from about 0.000001 mg/kg body weight to about 100 mg/kg body weight. A person of ordinary skill in the art would appreciate that dosage levels are based on a variety of factors, including the type of injury, the age, weight, sex, medical condition of the patient, the severity of the condition, the route of administration, and the particular active agent employed. Thus the dosage regimen may vary widely, but may be determined routinely by a physician using standard methods.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED50, which describes the dose in a particular dosage that is therapeutically effective for a particular disease manifestation in 50% of a population.

According to another embodiment, the therapeutic agent is an antimicrobial agent. According to another embodiment, the therapeutic agent is a cytokine. According to another embodiment, the therapeutic agent is a chemotherapeutic agent. According to another embodiment, the therapeutic agent is a non-sterodial anti-inflammatory agent. According to another embodiment, the therapeutic agent is a pharmaceutical composition.

The term “drug” as used herein refers to a therapeutic agent or any substance, other than food, used in the prevention, diagnosis, alleviation, treatment, or cure of disease.

The term “antimicrobial agent” as used herein refers to a natural or synthetic substance that kills microbes or inhibits them from growing and causing disease.

The term “cardiovascular injury” as used herein refers to an injury of, pertaining to, or affecting the heart and blood vessels.

The term “cerebrovascular injury” as used herein refers to an injury of, pertaining to, or affecting blood vessels in and to the brain.

The term “chemotherapeutic agent,” as used herein, refers to a chemical useful in the treatment or control of a disease. Non-limiting examples of chemotherapeutic agents suitable for the present invention include daunorubicin, doxorubicin, idarubicin, amrubicin, pirarubicin, epirubicin, mitoxantrone, etoposide, teniposide, vinblastine, mitomycin C, fluorouracil (5-FU), paclitaxel, docetaxel, actinomycin D, colchicines, topotecan, irinotecan, gemcitabine cyclosporine, verapamil, valspodor, probenecid, (E)-3-[[[3-[2-(7-chloro-2-quinolinyl)ethenyl]phenyl]-[[3-dimethylamino)-3-oxopropyl]thio]methyl]thio]-propanoic acid (MK571), N-(4-[2-(1,2,3,4-tetrahydro-6,7-dimethoxy-2-isoquinolyl)-ethyl]-phenyl-9,10-dihydro-5-methoxy-9-oxo-4-acridine carboxamide (elacridar, GF129918), zosuquidar trihydrochloride (LY335979), biricodar, terfenadine, quinidine, pervilleine A and tariquidar (XR9576).

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors. These receptors are located in the cell membrane and each allows a distinct signal transduction cascade to start in the cell, which eventually will lead to biochemical and phenotypic changes in the target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “inflammation” as used herein refers to a response to infection and injury in which cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue.

The term “microbial,” “microbe” or “microorganism,” as used herein, refers to an organism too small to be seen clearly with the naked eye, including, but not limited to, bacteria, fungi, molds, algae, protozoans, and viruses.

The term “LC₅₀” refers to the lethal concentration required to kill 50% of the test population measured in milligrams per liter. The term “LD₅₀” as used herein means a dose of a substance that produces death in 50% of a given population.

The term “non-steroidal anti-inflammatory agent” (“NSAID,” or “NAIDs”) as used herein, refers to drugs with analgesic, antipyretic (lowering an elevated body temperature and relieving pain without impairing consciousness) and, in higher doses, anti-inflammatory effects (reducing inflammation). The term “non-steroidal” is used to distinguish these drugs from steroids, which (among a broad range of other effects) have a similar eicosanoid-depressing, anti-inflammatory action. NSAIDs that are suitable for the compositions of the present invention include, but are not limited to, aspirin, ibruprofen, naproxen sodium, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acemetacin, fentiazac, zomepirac, clindanac, oxepiniac, felbinac, and ketorolac; fenamates, such as mefenamic, meclofenamic, flufenamic, niflumic, and tolfenamic acids; propionic acid derivatives, such as ibuprofen, naproxen, benoxaprofen, fluribiprofen, ketoprofen, fenoprofen, fenbufen, indopropfen, pirprofen, carpofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, trimethazone. Mixtures of non-steroidal anti-inflammatory agents also can be employed, as well as the pharmaceutically and/or dermatologically-acceptable salts and esters thereof.

The term “reduce” or “reducing” as used herein refers to the act of limiting the occurrence of the disorder in individuals at risk of developing a particular disorder.

The term “modulate” as used herein means to regulate, alter, adapt, or adjust to a certain measure or proportion.

The terms “inhibiting”, “inhibit” or “inhibition” as used herein are used to refer to reducing the amount or rate of a process, to stopping the process entirely, or to decreasing, limiting, or blocking the action or function thereof. Inhibition may include a reduction or decrease of the amount, rate, action function, or process by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99% when compared to a reference substance, wherein the reference substance is a substance that is not inhibited.

The term “treat” or “treating” as used herein refers to accomplishing one or more of the following: (a) reducing the severity of a disorder; (b) limiting the development of symptoms characteristic of a disorder being treated; (c) limiting the worsening of symptoms characteristic of a disorder being treated; (d) limiting the recurrence of a disorder in patients that previously had the disorder; and (e) limiting recurrence of symptoms in patients that were previously symptomatic for the disorder.

The term “disease” or “disorder” as used herein refers to an impairment of health or a condition of abnormal functioning.

The term “syndrome” as used herein refers to a pattern of symptoms indicative of some disease or condition.

The term “injury” as used herein refers to damage or harm to a structure or function of the body caused by an outside agent or force, which may be physical or chemical.

The term “condition” as used herein refers to a variety of health states and is meant to include disorders or diseases caused by any underlying mechanism, disorder or injury, and the promotion of healthy tissues and organs.

The term “administering” which as used herein refers to causing to take, give or apply, includes in vivo administration, as well as administration directly to tissue ex vivo. Generally, compositions may be administered systemically either orally, buccally, parenterally, topically, by inhalation or insufflation (i.e., through the mouth or through the nose), or rectally in dosage unit formulations containing conventional nontoxic pharmaceutically acceptable carriers, adjuvants, and vehicles as desired, or may be locally administered by means such as, but not limited to, injection, implantation, grafting, topical application, or parenterally.

The term “parenteral” as used herein refers to introduction into the body by way of an injection (i.e., administration by injection), including, for example, subcutaneously (i.e., an injection beneath the skin), intramuscularly (i.e., an injection into a muscle); intravenously (i.e., an injection into a vein), intrathecally (i.e., an injection into the space around the spinal cord or under the arachnoid membrane of the brain), intrasternal injection, or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

The term “topical” refers to administration of a composition at, or immediately beneath, the point of application. The phrase “topically applying” describes application onto one or more surfaces(s) including epithelial surfaces. Although topical administration, in contrast to transdermal administration, generally provides a local rather than a systemic effect the terms “topical administration” and “transdermal administration” as used herein, unless otherwise stated or implied, are used interchangeably.

### 4.1. Embodiments Wherein the Therapeutic Agent is a Biological Agent

### 4.1.1. Cells

According to another embodiment, the therapeutic agent is a biological agent. According to some embodiments, the biological agent is a cell. According to some embodiments, the biocompatible biogel further comprises cells. The biocompatible biogel may be used as a delivery and tissue incorporation matrix to deliver differentiated stem cell populations, adult cell populations or tissue components. Examples of such applications include, but are not limited to, cerebrovascular, cardiovascular, neural, orthopedic (bone and cartilage), transplant or tissue regeneration/engineering applications.

According to some embodiments, the cells are stem cells.

As used herein, the term “stem cells” refers to undifferentiated cells having high proliferative potential with the ability to self-renew that can migrate to areas of injury and can generate daughter cells that can undergo terminal differentiation into more than one distinct cell phenotype. These cells have the ability to differentiate into various cells types and thus promote the regeneration or repair of a diseased or damaged tissue of interest.

Specialized protein receptors that have the capability of selectively binding or adhering to other signaling molecules coat the surface of every cell in the body. Cells use these receptors and the molecules that bind to them as a way of communicating with other cells and to carry out their proper functions in the body. Each cell type has a certain combination of receptors, or markers, on their surface that makes them distinguishable from other kinds of cells.

Stem cell markers are given short-hand names based on the molecules that bind to the corresponding stem cell surface receptors. In many cases, a combination of multiple markers is used to identify a particular stem cell type. Researchers often identify stem cells in shorthand by a combination of marker names reflecting the presence (+) or absence (−) of them. For example, a special type of hematopoietic stem cell from blood and bone marrow called “side population” or “SP” is described as (CD34^{-/low}, c-Kit⁺, Sca-1⁺).

The following markers commonly are used by skilled artisans to identify stem cells and to characterize differentiated cell types (http://stemcells.nih.gov/info/scireport/appendixE.asp#eii:

| | | |
|---|---|---|
| Marker Name | Cell Type | Significance |

Blood Vessel

| | | |
|---|---|---|
| Fetal liver kinase-1 | Endothelial | Cell-surface receptor protein that identifies endothelial cell progenitor; |
| (Flk1) | | marker of cell-cell contacts |
| Smooth muscle cell- | Smooth muscle | Identifies smooth muscle cells in the wall of blood vessels |
| specific myosin heavy | | |
| chain | | |
| Vascular endothelial | Smooth muscle | Identifies smooth muscle cells in the wall of blood vessels |
| cell cadherin | | |

Bone

| | | |
|---|---|---|
| Bone-specific alkaline | Osteoblast | Enzyme expressed in osteoblast; activity indicates bone formation |
| phosphatase (BAP) | | |
| Hydroxyapatite | Osteoblast | Minerlized bone matrix that provides structural integrity; marker of bone |
| | | formation |
| Osteocalcin (OC) | Osteoblast | Mineral-binding protein uniquely synthesized by osteoblast; marker of |
| | | bone formation |

Bone Marrow and Blood

| | | |
|---|---|---|
| Bone morphogenetic | Mesenchymal stem and | Important for the differentiation of committed mesenchymal cell types |
| protein receptor | progenitor cells | from mesenchymal stem and progenitor cells; BMPR identifies early |
| (BMPR) | | mesenchymal lineages (stem and progenitor cells) |
| CD4 and CD8 | White blood cell (WBC) | Cell-surface protein markers specific for mature T lymphocyte (WBC |
| | | subtype) |
| CD34 | Hematopoietic stem cell | Cell-surface protein on bone marrow cell, indicative of a HSC and |
| | (HSC), satellite, | endothelial progenitor; CD34 also identifies muscle satellite, a muscle |
| | endothelial progenitor | stem cell |
| CD34⁺Sca1⁺ Lin⁻ | Mesencyhmal stem cell | Identifies MSCs, which can differentiate into adipocyte, osteocyte, |
| profile | (MSC) | chondrocyte, and myocyte |
| | Absent on HSC | |
| CD38 | Present on WBC lineages | Cell-surface molecule that identifies WBC lineages. Selection of |
| | | CD34⁺/CD38⁻ cells allows for purification of HSC populations |
| CD44 | Mesenchymal | A type of cell-adhesion molecule used to identify specific types of |
| | | mesenchymal cells |
| c-Kit | HSC, MSC | Cell-surface receptor on BM cell types that identifies HSC and MSC; |
| | | binding by fetal calf serum (FCS) enhances proliferation of ES cells, |
| | | HSCs, MSCs, and hematopoietic progenitor cells |
| Colony-forming unit | HSC, MSC progenitor | CFU assay detects the ability of a single stem cell or progenitor cell to |
| (CFU) | | give rise to one or more cell lineages, such as red blood cell (RBC) and/or |
| | | white blood cell (WBC) lineages |
| Fibroblast colony- | Bone marrow fibroblast | An individual bone marrow cell that has given rise to a colony of |
| forming unit (CFU-F) | | multipotent fibroblastic cells; such identified cells are precursors of |
| | | differentiated mesenchymal lineages |
| Hoechst dye | Absent on HSC | Fluorescent dye that binds DNA; HSC extrudes the dye and stains lightly |
| | | compared with other cell types |
| Leukocyte common | WBC | Cell-surface protein on WBC progenitor |
| antigen (CD45) | HSC, MSC | |
| Lineage surface | Differentiated RBC and | Thirteen to 14 different cell-surface proteins that are markers of mature |
| antigen (Lin) | WBC lineages | blood cell lineages; detection of Lin-negative cells assists in the |
| | | purification of HSC and hematopoietic progenitor populations |
| Mac-1 | WBC | Cell-surface protein specific for mature granulocyte and macrophage |
| | | (WBC subtypes) |
| Muc-18 (CD146) | Bone marrow fibroblasts, | Cell-surface protein (immunoglobulin superfamily) found on bone marrow |
| | endothelial | fibroblasts, which may be important in hematopoiesis; a subpopulation of |
| | | Muc-18+ cells are mesenchymal precursors |
| Stem cell antigen | HSC, MSC | Cell-surface protein on bone marrow (BM) cell, indicative of HSC and |
| (Sca-1) | | MSC Bone Marrow and Blood cont. |
| Stro-1 antigen | Stromal (mesenchymal) | Cell-surface glycoprotein on subsets of bone marrow stromal |
| | precursor cells, | (mesenchymal) cells; selection of Stro-1+ cells assists in isolating |
| | hematopoietic cells | mesenchymal precursor cells, which are multipotent cells that give rise to |
| | | adipocytes, osteocytes, smooth myocytes, fibroblasts, chondrocytes, and |
| | | blood cells |
| Thy-1 | HSC, MSC | Cell-surface protein; negative or low detection is suggestive of HSC |

Cartilage

| | | |
|---|---|---|
| Collagen types II and | Chondrocyte | Structural proteins produced specifically by chondrocyte |
| IV | | |
| Keratin | Keratinocyte | Principal protein of skin; identifies differentiated keratinocyte |
| Sulfated proteoglycan | Chondrocyte | Molecule found in connective tissues; synthesized by chondrocyte |

Fat

| | | |
|---|---|---|
| Adipocyte lipid- | Adipocyte | Lipid-binding protein located specifically in adipocyte |
| binding protein | | |
| (ALBP) | | |
| Fatty acid transporter | Adipocyte | Transport molecule located specifically in adipocyte |
| (FAT) | | |
| Adipocyte lipid- | Adipocyte | Lipid-binding protein located specifically in adipocyte |
| binding protein | | |
| (ALBP) | | |

General

| | | |
|---|---|---|
| Y chromosome | Male cells | Male-specific chromosome used in labeling and detecting donor cells in |
| | | female transplant recipients |
| Karyotype | Most cell types | Analysis of chromosome structure and number in a cell |

Liver

| | | |
|---|---|---|
| Albumin | Hepatocyte | Principal protein produced by the liver; indicates functioning of maturing |
| | | and fully differentiated hepatocytes |
| B-1 integrin | Hepatocyte | Cell-adhesion molecule important in cell-cell interactions; marker |
| | | expressed during development of liver |

Nervous System

| | | |
|---|---|---|
| CD133 | Neural stem cell, HSC | Cell-surface protein that identifies neural stem cells, which give rise to |
| | | neurons and glial cells |
| Glial fibrillary acidic | Astrocyte | Protein specifically produced by astrocyte. |
| protein (GFAP) | | |
| Microtubule- | Neuron | Dendrite-specific MAP; protein found specifically in dendritic branching |
| associated protein-2 | | of neuron |
| (MAP-2) | | |
| Myelin basic protein | Oligodendrocyte | Protein produced by mature oligodendrocytes; located in the myelin sheath |
| (MPB) | | surrounding neuronal structures |
| Nestin | Neural progenitor | Intermediate filament structural protein expressed in primitive neural |
| | | tissue |
| Neural tubulin | Neuron | Important structural protein for neuron; identifies differentiated neuron |
| Neurofilament (NF) | Neuron | Important structural protein for neuron; identifies differentiated neuron |
| Neurosphere | Embryoid body (EB), ES | Cluster of primitive neural cells in culture of differentiating ES cells; |
| | | indicates presence of early neurons and glia |
| Noggin | Neuron | A neuron-specific gene expressed during the development of neurons |
| O4 | Oligodendrocyte | Cell-surface marker on immature, developing oligodendrocyte |
| O1 | Oligodendrocyte | Cell-surface marker that characterizes mature oligodendrocyte |
| Synaptophysin | Neuron | Neuronal protein located in synapses; indicates connections between |
| | | neurons |
| Tau | Neuron | Type of MAP; helps maintain structure of the axon |

Pancreas

| | | |
|---|---|---|
| Cytokeratin 19 | Pancreatic epithelium | CK19 identifies specific pancreatic epithelial cells that are progenitors for |
| (CK19) | | islet cells and ductal cells |
| Glucagon | Pancreatic islet | Expressed by alpha-islet cell of pancreas |
| Insulin | Pancreatic islet | Expressed by beta-islet cell of pancreas Pancreas |
| Insulin-promoting | Pancreatic islet | Transcription factor expressed by beta-islet cell of pancreas |
| factor-1 (PDX-1) | | |
| Nestin | Pancreatic progenitor | Structural filament protein indicative of progenitor cell lines including |
| | | pancreatic |
| Pancreatic polypeptide | Pancreatic islet | Expressed by gamma-islet cell of pancreas |
| Somatostatin | Pancreatic islet | Expressed by delta-islet cell of pancreas |

Pluripotent Stem Cells

| | | |
|---|---|---|
| Alkaline phosphatase | Embryonic stem (ES), | Elevated expression of this enzyme is associated with undifferentiated |
| | embryonal carcinoma | pluripotent stem cell (PSC) |
| | (EC) | |
| Alpha-fetoprotein | Endoderm | Protein expressed during development of primitive endoderm; reflects |
| (AFP) | | endodermal differentiation Pluripotent Stem Cells |
| Bone morphogenetic | Mesoderm | Growth and differentiation factor expressed during early mesoderm |
| protein-4 | | formation and differentiation |
| Brachyury | Mesoderm | Transcription factor important in the earliest phases of mesoderm |
| | | formation and differentiation; used as the earliest indicator of mesoderm |
| | | formation |
| Cluster designation 30 | ES, EC | Surface receptor molecule found specifically on PSC |
| (CD30) | | |
| Cripto (TDGF-1) | ES, cardiomyocyte | Gene for growth factor expressed by ES cells, primitive ectoderm, and |
| | | developing cardiomyocyte |
| GATA-4 gene | Endoderm | Expression increases as ES differentiates into endoderm |
| GCTM-2 | ES, EC | Antibody to a specific extracellular-matrix molecule that is synthesized by |
| | | undifferentiated PSCs |
| Genesis | ES, EC | Transcription factor uniquely expressed by ES cells either in or during the |
| | | undifferentiated state of PSCs |
| Germ cell nuclear | ES, EC | Transcription factor expressed by PSCs |
| factor | | |
| Hepatocyte nuclear | Endoderm | Transcription factor expressed early in endoderm formation |
| factor-4 (HNF-4) | | |
| Nestin | Ectoderm, neural and | Intermediate filaments within cells; characteristic of primitive |
| | pancreatic progenitor | neuroectoderm formation |
| Neuronal cell- | Ectoderm | Cell-surface molecule that promotes cell-cell interaction; indicates |
| adhesion molecule (N- | | primitive neuroectoderm formation |
| CAM) | | |
| OCT4/POU5F1 | ES, EC | Transcription factor unique to PSCs; essential for establishment and |
| | | maintenance of undifferentiated PSCs |
| Pax6 | Ectoderm | Transcription factor expressed as ES cell differentiates into |
| | | neuroepithelium |
| Stage-specific | ES, EC | Glycoprotein specifically expressed in early embryonic development and |
| embryonic antigen-3 | | by undifferentiated PSCs |
| (SSEA-3) | | |
| Stage-specific | ES, EC | Glycoprotein specifically expressed in early embryonic development and |
| embryonic antigen-4 | | by undifferentiated PSCs |
| (SSEA-4) | | |
| Stem cell factor (SCF | ES, EC, HSC, MSC | Membrane protein that enhances proliferation of ES and EC cells, |
| or c-Kit ligand) | | hematopoietic stem cell (HSCs), and mesenchymal stem cells (MSCs); |
| | | binds the receptor c-Kit |
| Telomerase | ES, EC | An enzyme uniquely associated with immortal cell lines; useful for |
| | | identifying undifferentiated PSCs |
| TRA-1-60 | ES, EC | Antibody to a specific extracellular matrix molecule is synthesized by |
| | | undifferentiated PSCs |
| TRA-1-81 | ES, EC | Antibody to a specific extracellular matrix molecule normally synthesized |
| | | by undifferentiated PSCs |
| Vimentin | Ectoderm, neural and | Intermediate filaments within cells; characteristic of primitive |
| | pancreatic progenitor | neuroectoderm formation |

Skeletal Muscle/Cardiac/Smooth Muscle

| | | |
|---|---|---|
| MyoD and Pax7 | Myoblast, myocyte | Transcription factors that direct differentiation of myoblasts into mature |
| | | myocytes |
| Myogenin and MR4 | Skeletal myocyte | Secondary transcription factors required for differentiation of myoblasts |
| | | from muscle stem cells |
| Myosin heavy chain | Cardiomyocyte | A component of structural and contractile protein found in cardiomyocyte |
| Myosin light chain | Skeletal myocyte | A component of structural and contractile protein found in skeletal |
| | | myocyte |

According to some embodiments, the cells of interest are progenitor cells. The term “progenitor cell” as used herein refers to an immature or undifferentiated cell that may be activated (meaning to stimulate the cell to proliferate and differentiate) by growing suspensions of the cells with added growth factors. Progenitor cells are referred to as colony-forming units (CFU) or colony-forming cells (CFC). The specific lineage of a progenitor cell is indicated by a suffix, such as, but not limited to, CFU-F (fibroblastic).

According to some embodiments, the cells of interest are differentiated cells. The term “cellular differentiation” as used herein refers to the process by which cells acquire a cell type.

According to another embodiment, the cells of interest are chondrocytes. The term “chondrocytes” as used herein refers to cells found in cartilage that produce and maintain the cartilaginous matrix. From least to terminally differentiated, the chondrocytic lineage is (i) Colony-forming unit-fibroblast (CFU-F); (ii) mesenchymal stem cell/marrow stromal cell (MSC); and (iii) chondrocyte. As used herein, the terms “osteoprogenitor cells,” “mesenchymal cells,” “mesenchymal stem cells (MSC),” or “marrow stromal cells” are used interchangeably to refer to multipotent stem cells that differentiate from CFU-F cells, and are capable of differentiating along several lineage pathways into osteoblasts, chondrocytes, myocytes and adipocytes. The term “chondrogenesis” refers to the formation of new cartilage from cartilage forming or chondrocompetent cells.

According to some embodiments, the cells of interest are used for repair or regeneration of a damaged or diseased tissue or organ. In some such embodiments, the cells of interest are autologous, meaning that the cells are reimplanted into the individual from whom they were obtained. In some such embodiments, the cells of interest are allogenic, meaning that they are transplanted from an individual different from the individual from whom they were obtained.

According to some embodiments, the cells of interest replace or supplement a function in cells in a tissue or organ, which function is missing or deficient when compared to normal as a consequence of disease or genetics.

According to some such embodiments biocompatible biogel composition is used to deliver stem cells to repair a cerebrovascular injury.

According to some such embodiments the biocompatible biogel composition is used to deliver stem cells to repair a cardiovascular injury. In some such embodiments, the present invention provides a method to treat myocardial injury due to myocardial infarction.

### 4.1.2. Embodiments Wherein the Therapeutic Agent is a Polypeptide

According to another embodiment, the present invention provides a composition comprising a biocompatible biogel comprising a polypeptide comprising a sequence according to general formula I:

Z1-X1-X2-X3-X4 X5-X6-X7-X8-X9-X10-Z2 [Formula I]

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid.

According to another embodiment, the biocompatible composition comprises a biocompatible biogel comprising a polypeptide having an amino acid sequence according to general formula I:

Z1-X1-X2-X3-X4 X5-X6-X7-X8-X9-X10-Z2 [Formula I]

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid;
wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

As used herein, “sequence identity” or “identity” in the context of two nucleic acid or polypeptide sequences refers to the residues in the two sequences which are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The terms “substantial identity” in the context of a peptide indicates that a peptide comprises a sequence with at least 70% sequence identity to a reference sequence, at least 80%, at least 85%, at least 90% or 95% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

The polypeptides having an amino acid sequence of the general formula I are isolated molecules. An isolated molecule is a molecule that is substantially pure and is free of other substances with which it is ordinarily found in nature or in vivo systems to an extent practical and appropriate for its intended use. In particular, the polypeptides having an amino acid sequence of the general formula I are sufficiently pure and are sufficiently free from other biological constituents of host cells so as to be useful in, for example, producing pharmaceutical preparations or sequencing. Because the polypeptides having an amino acid sequence of the general formula I may be admixed with a pharmaceutically-acceptable carrier in a pharmaceutical preparation, these polypeptides may comprise only a small percentage by weight of the preparation. The polypeptide having an amino acid sequence of the general formula I is nonetheless substantially pure in that it has been substantially separated from the substances with which it may be associated in living systems or during synthesis.

According to another embodiment, the therapeutic agent is an isolated polypeptide having an amino acid sequence according to general formula I, where X4 is R, X5 is Q and X8 is V. According another embodiment, the therapeutic agent is an isolated polypeptide having an amino acid sequence of KAFAKLAARLYRKALARQLGVAA [SEQ ID NO: 1]. According to another embodiment, the therapeutic agent is an isolated polypeptide having an amino acid sequence of FAKLAARLYRKALARQLGVAA [SEQ ID NO: 2].

In some embodiments, the therapeutic agent modulates Tumor Necrosis Factor alpha (“TNF-α”) secretion through the targeting of regulatory proteins and modulators of the secretory pathway for TNF-α. Target proteins may include, but are not limited to, syntaxin4, munc18c, Cdc42, Rac-1, VAMP3, syntaxin3, syntaxin6, Vtil-B, Vap3, RGS16 and RGSGAIP. TNF-α is one of the main cytokines released from activated macrophages at sites of inflammation. TNF-α is an important pro-inflammatory mediator that primes the immune system by activating and recruiting other cells. At sites of extensive or persistent inflammation, TNF-α is often secreted in excess by large numbers of activated macrophages.

Resting macrophages have low O₂ consumption and little or no cytokine secretion. Upon activation by an appropriate stimulus, macrophages undergo many changes to enact tumor cytotoxic or microbicidal actions. Amongst the functions initiated during activation is the synthesis and secretion of cytokines including TNF-α.

TNF-α is synthesized in macrophages as a 26 kD Type II transmembrane precursor which accumulates in the Golgi complex, TNF-α is then trafficked from the Golgi complex to the cell surface where a 17 kD ectodomain is cleaved off by the enzyme TACE. Trimers of this soluble subunit then form the circulating cytokine. TNF-α also can be retained on the macrophage surface in an uncleaved form.

Excessive secretion of TNF-α has been implicated in several pathologies associated with acute and chronic inflammatory diseases (see, for example, Beutler, 1999; J. Rheumatol., 26:16-21; Vassalli, 1992, Annu. Rev. Immunol., 10:411-452). Excessive or inappropriate secretion of TNF-α is one of the leading causes of death in acute conditions such as septic shock, and is one of the main factors contributing to ongoing tissue damage in chronic inflammatory diseases such as inflammatory bowel disease (IBS), arthritis, psoriasis, congestive heart disease, and chronic obstructive pulmonary disease.

According to another embodiment, the therapeutic agent is an isolated polypeptide having at least 90% amino acid sequence identity to KAFAKLAARLYRKALARQLGVAA [SEQ ID NO: 1], wherein the polypeptide inhibits TNF-α secretion.

According to another embodiment, the therapeutic agent is an isolated polypeptide having at least 90% amino acid sequence identity to identity to FAKLAARLYRKALARQLGVAA [SEQ ID NO: 2], wherein the polypeptide inhibits TNF-α secretion.

According to another embodiment, the present invention provides an isolated nucleic acid that encodes a polypeptide having at least 90% amino acid sequence identity to KAFAKLAARLYRKALARQLGVAA [SEQ ID NO: 1], wherein the polypeptide inhibits TNF-α secretion. According to another embodiment, the present invention provides an isolated nucleci acid that encodes a polypeptide having at least 90% amino acid sequence identity to FAKLAARLYRKALARQLGVAA [SEQ ID NO: 2], wherein the polypeptide inhibits TNF-α secretion.

According to another embodiment, the present invention provides an isolated nucleic acid that specifically hybridizes to mRNA encoding a peptide having an amino acid sequence of KAFAKLAARLYRKALARQLGVAA [SEQ ID NO: 1], wherein the polypeptide inhibits TNF-α secretion. According to another embodiment, the present invention provides an isolated nucleic acid that specifically hybridizes to mRNA encoding a peptide having an amino acid sequence of FAKLAARLYRKALARQLGVAA [SEQ ID NO: 2], wherein the polypeptide inhibits TNF-α secretion. The term “specifically hybridizes” as used herein refers to the process of a nucleic acid distinctively or definitively forming base pairs with complementary regions of at least one strand of DNA that originally was not paired to the nucleic acid. For example, a nucleic acid that may bind or hybridize to at least a portion of an mRNA of a cell encoding a peptide having an amino acid sequence of KAFAKLAARLYRKALARQLGVAA [SEQ ID NO: 1] or FAKLAARLYRKALARQLGVAA [SEQ ID NO: 2] may be considered a nucleic acid that specifically hybridizes. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 90% sequence identity, or at least 100% sequence identity (i.e., complementary) with each other.

Methods of extraction of RNA are well-known in the art and are described, for example, in J. Sambrook et al., “Molecular Cloning: A Laboratory Manual” (Cold Spring Harbor Laboratory Press, Cold Spring Harbor, N.Y., 1989), vol. 1, ch. 7, “Extraction, Purification, and Analysis of Messenger RNA from Eukaryotic Cells,” incorporated herein by this reference. Other isolation and extraction methods also are well-known, for example in F. Ausubel et al., “Current Protocols in Molecular Biology”, John Wiley & Sons, 2007). Typically, isolation is performed in the presence of chaotropic agents, such as guanidinium chloride or guanidinium thiocyanate, although other detergents and extraction agents alternatively may be used. Typically, the mRNA is isolated from the total extracted RNA by chromatography over oligo(dT)-cellulose or other chromatographic media that have the capacity to bind the polyadenylated 3′-portion of mRNA molecules. Alternatively, but less preferably, total RNA can be used. However, it generally is preferred to isolate poly(A)+RNA from mammalian sources.

According to another embodiment, the present invention provides an antibody or an antibody fragment that specifically binds to an amino acid sequence of KAFAKLAARLYRKALARQLGVAA [SEQ ID NO: 1], or fragment thereof.

According to another embodiment, the present invention provides an antibody or an antibody fragment that specifically binds to an amino acid sequence of FAKLAARLYRKALARQLGVAA [SEQ ID NO: 2], or fragment thereof.

According to another embodiment, the therapeutic agent is a cationic peptide such as those described in U.S. Provisional Application No. 60/994,970, titled “Polypeptide Inhibitors of Kinases and Uses Thereof,” and those described in U.S. Provisional Application No. 60/963,941, titled “Kinase Inhibitors and Uses Therefor,” both of which are incorporated in their entirety herein by reference.

### 4.1.3. Embodiments Wherein the Therapeutic Agent is a Growth Factor

According to some embodiments, the therapeutic agent is a growth factor. Growth factors include, but are not limited to, vascular endothelial growth factor (VEGF), fibroblast growth factor (FGF), bone morphogenic protein (BMP), transforming growth factor (TGF), nerve growth factor (NGF), neurotrophic factor 3 (NT3), platelet derived growth factor (PDGF), and brain derived neurotrophic factor (BDNF).

The term “VEGF-1” or “vascular endothelial growth factor-1” refers to a cytokine that mediates numerous functions of endothelial cells including proliferation, migration, invasion, survival, and permeability. VEGF is critical for angiogenesis. As used herein, the term “angiogenesis” refers to the process of formation and development of blood vessels.

The term “Fibroblast Growth Factors” (FGFs) refers to a family of signaling molecules involved in angiogenesis, wound healing, and embryonic development. The FGFs are heparin-binding proteins; interactions with cell-surface associated heparan sulfate proteoglycans have been shown to be essential for FGF signal transduction.

Bone Morphogenic Protein (BMP) refers to a superfamily of proteins that promote the formation of bone and the skeleton and help mend broken bones. BMP1 is not closely related to other known growth factors. Other members of the BMP family, which are numbered starting from BMP2, belong to a superfamily called transforming growth factor beta (TGF-β).

The terms “Transforming Growth Factor”, “tumor growth factor” or “TGF” are used interchangeably to describe two classes of polypeptide growth factors, TGFα and TGFβ. TGFα, which is upregulated in some human cancers, is produced in macrophages, brain cells, and keratinocytes, and induces epithelial development. TGFβ exists in three known subtypes in humans, TGFβ1, TGFβ2, and TGFβ3 that are upregulated in some human cancers, and play crucial roles in tissue regeneration, cell differentiation, embryonic development, and regulation of the immune system. TGFβ receptors are single pass serine/threonine kinase receptors.

The term “Nerve Growth Factor” (NGF) refers to a small secreted protein that induces the differentiation and survival of particular target neurons (nerve cells).

The term “neurotrophin” refers to a family of chemicals that help to stimulate and control neurogenesis. The terms “Neurotrophin 3” or “neurotrophic factor 3” (NT3) are used interchangeably to refer to a neurotrophic protein that acts on certain neurons of the peripheral and central nervous system to help support the survival and differentiation of existing neurons, and encourages the growth and differentiation of new neurons and synapses. The term “Brain Derived Neurotrophic Factor” (BDNF) refers to another neurotrophic protein found in a range of tissue and cell types that acts on certain neurons of the central nervous system and the peripheral nervous system to help support the survival of existing neurons and encourage the growth and differentiation of new neurons and synapses.

The term “platelet derived growth factor” (PDGF) refers to a protein, produced by platelets and other cells, that strongly stimulates cell growth and division and is involved in normal wound healing.

The present invention includes active portions, fragments, derivatives, mutants, and functional variants of polypeptides to the extent such active portions, fragments, derivatives, and functional variants retain any of the biological properties of the polypeptide. An “active portion” of a polypeptide means a peptide that is shorter than the full length polypeptide, but which retains measurable biological activity. In some embodiments, a “fragment” of a polypeptide refers to a stretch of amino acid residues of at least five to seven contiguous amino acids; in some embodiments, a “fragment” of a polypeptide refers to at least about seven to nine contiguous amino acids; in some embodiments, a “fragment” of a polypeptide refers to at least about nine to thirteen contiguous amino acids; and in some embodiments, a “fragment” of a polypeptide refers to at least about twenty to thirty or more contiguous amino acids. A “derivative” of a polypeptide or a fragment thereof means a polypeptide modified by varying the amino acid sequence of the protein, for example, by manipulating the nucleic acid encoding the protein or by altering the protein itself. Such derivatives of the natural amino acid sequence may involve insertion, addition, deletion, or substitution of one or more amino acids, and may or may not alter the essential activity of the original polypeptide.

### 4.2. Embodiments Wherein the Therapeutic Agent is a Pharmaceutical Composition

According to another embodiment, the therapeutic agent is a pharmaceutical composition. The pharmaceutical composition also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, microencapsulated, and if appropriate, with one or more excipients, encochleated (meaning “trapped”), coated onto microscopic gold particles, contained in liposomes, pellets for implantation into the tissue, or dried onto an object to be rubbed into the tissue. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer 1990 Science 249, 1527-1533, which is herein by reference incorporated in its entirety.

The polypeptides having an amino acid sequence of general formula I, and optionally other therapeutic agents, may be combined with the biogel components per se (neat) or in the form of a pharmaceutically acceptable salt. When used in medicine the salts should be pharmaceutically acceptable, but non-pharmaceutically acceptable salts conveniently may be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present invention or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products thereby are obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts may be prepared in situ during the final isolation and purification of compounds described within the invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids also may be made.

The formulations may be prepared by any of the methods well known in the art of pharmacy. All methods include the step of bringing into association the therapeutic agent, or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent or a pharmaceutically acceptable ester, salt, solvate or prodrug thereof may be mixed with other active materials that do not impair the desired action, or with materials that supplement the desired action.

These compositions may also contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth; and mixtures thereof.

Alternately, pharmaceutical composition formulations may be made by forming microencapsulated matrices of the drug in biodegradable polymers such as, but not limited to, polylactide-polyglycolide. Depending upon the ratio of drug to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include, but are not limited to, poly(orthoesters) and poly(anhydrides). Depot injectable formulations also are prepared by entrapping the drug in liposomes or microemulsions, which are compatible with body tissues.

Aqueous formulations may be sterilized, for example, by filtration through a bacterial-retaining filter or by incorporating sterilizing agents in the form of sterile solid compositions that may be dissolved or dispersed in sterile water or other sterile medium. Sterile aqueous or oleaginous suspensions may be formulated according to the known art using suitable dispersing or wetting agents and suspending agents. The sterile preparation also may be a sterile solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed either as a solvent or suspending medium. For this purpose any bland fixed oil may be employed including synthetic mono- or diglycerides. In addition, fatty acids such as oleic acid are used in the preparation of such formulations.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

According to some embodiments, the therapeutic agent is embedded (meaning enclosed firmly in) the surrounding biogel composition. In some embodiments, the therapeutic agent is encapsulated (meaning that the gel-like system of the present invention forms a case, envelope or covering for the therapeutic). All FDA approved therapeutics (i.e., therapeutic agents) may be encapsulated by the biogel composition of the present invention. Examples of such encapsulated therapeutic agents include, but are not limited to, nonsteroidal anti-inflammatory (NSAID), growth factors, therapeutic peptides, kinase inhibitors, phosphodiesterase inhibitors, antibodies, antimicrobial agents, and chemotherapeutics.

The pharmaceutical compositions described within the present invention contain a therapeutically effective amount of a therapeutic agent and optionally other therapeutic agents included in a pharmaceutically-acceptable carrier. The term “pharmaceutically-acceptable carrier” as used herein refers to one or more compatible solid or liquid filler, diluents or encapsulating substances, which are suitable for administration to a human or other vertebrate animal. The term “carrier” as used herein refers to an organic or inorganic ingredient, natural or synthetic, with which the active ingredient is combined to facilitate the application. The active ingredient may be a polypeptide having an amino acid sequence of general formula I. The components of the pharmaceutical compositions also are capable of being comingled in a manner such that there is no interaction that would substantially impair the desired pharmaceutical efficiency.

The therapeutic agent(s), including polypeptides having an amino acid sequence of general formula I, may be provided in particles. The term “particle” as used herein refers to any discrete unit of material structure. Particles may include nanoparticles or microparticles (or in some instances larger) that may contain in whole or in part the therapeutic agent. As used herein, the term “microparticle” refers to a particle having a diameter of about 1 micron to about 1000 microns. As used herein, the term “nanoparticle” refers to a particle having a diameter of about 1 nanometer to about 1000 nanometers. The particles may contain the therapeutic agent(s) in a core surrounded by a coating. The therapeutic agent(s) also may be dispersed throughout the particles. The therapeutic agent(s) also may be adsorbed on at least one surface of the particles. The particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain a therapeutic agent in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

According to some embodiments, a particle, nanoparticle or microparticle according to the present invention may be composed of a degradable polyester, including, but not limited to, polymers and co-polymers of polylactide, polyglycolide, and polycaprolactone. In some embodiments, the nanoparticle or microparticle may be composed of other degradable polymers, including, but not limited to, polyanhydrides, poly(ortho-esters), proteins, polynucleotides, and polyacrylamides. In some embodiments, the nanoparticle or microparticle may be composed of other erodible (meaning capable of eroding (disintegrating)) polymers, including, but not limited to alginate, agarose, dextran, and chitosan.

The therapeutic agent(s) may be contained in controlled release systems. In order to prolong the effect of a drug, it often is desirable to slow the absorption of the drug. This may be accomplished by the use of a liquid suspension of crystalline or amorphous material with poor water solubility. The rate of absorption of the drug then depends upon its rate of dissolution which, in turn, may depend upon crystal size and crystalline form. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. Alternatively, delayed absorption of a parenterally administered drug form is accomplished by dissolving or suspending the drug in an oil vehicle. The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. The term “long-term” release, as used herein, means that the implant is constructed and arranged to deliver therapeutic levels of the active ingredient for at least 7 days, and preferably about 30 days to about 60 days. Long-term sustained release implants are well-known to those of ordinary skill in the art and include some of the release systems described above.

According to some embodiments, the biocompatible biogel composition further comprises a slow-release extracellular matrix agent. According to some such embodiments, the extracellular matrix agent is a chondroitinase or a hyaluronidase.

According to some embodiments, the biocompatible biogel composition stabilizes short half-lived drugs by binding them to matrix components, and thereby extending the delivery period without need for reapplication. Examples of such applications include, but are not limited to, periodontal and/or surgical pain relief; pain/inflammation reduction in conjunction with orthopedic injury; surgical procedures; autoimmune disorders; inflammatory disorders; new life-cycle extension/formulation for existing brands; or to improve-upon delivery characteristics of existing generic drugs.

### 4.3. Combinations of Therapeutic Agents

According to some embodiments, the biocompatible biogel comprises a therapeutic agent combined with other therapeutic agents. The therapeutic agents may be combined simultaneously or sequentially. When other therapeutic agents are combined simultaneously, they may be combined in the same or separate formulations, as long as the formulations are biocompatible.

### 5. Biogel Formation

Biocompatible biogels comprise a physical network based on polymers, peptides, and polysaccharides. When these peptide-polymer molecules are mixed with polysaccharides, affinity-based interactions between the peptides and polysaccharides facilitate the formation of a physical gel-like material. In general, precursor components of the biocompatible biogel may be combined in a flowable composition with a delayed crosslinking chemistry to make a crosslinked material in situ. Optionally, the biocompatible biogel further comprises a therapeutic agent that is released over a suitable period of time. The crosslinking reactions generally occur in aqueous solution under physiological conditions. Generally, the crosslinking reactions do not release heat of polymerization or require exogenous energy sources for initiation or to trigger polymerization. In the case of injected materials, the viscosity may be controlled so that the material is introduced through a small diameter catheter or needle. Viscosity further may be controlled to keep precursors in place until they form a gel so that the precursors do not diffuse away from the intended site of use.

According to some embodiments, the biocompatible biogel may be low-swelling, as measurable by the biogel having a weight increasing no more than about 0% to about 10% or to about 50% upon exposure to a physiological solution for twenty-four hours relative to a weight of the biogel at the time of formation. One skilled in the art immediately will appreciate that all ranges and values within or otherwise relating to these explicitly articulated limits are disclosed herein. Unless otherwise indicated, swelling of a biogel relates to its change in volume (or weight) between the time of its formation when crosslinking is effectively complete and the time after being placed in vitro as a physiological solution in an unconstrained state for twenty-four hours, at which point it may be reasonably assumed to have achieved its equilibrium swelling state. For most embodiments, crosslinking effectively is complete within no more than about fifteen minutes such that the initial weight generally may be noted at about 15 minutes after formation as Weight at initial formation. Accordingly, this formula is used: % swelling=[(Weight at 24 hours−Weight at initial formation)/Weight at initial formation]*100. In the case of biogels that have substantial degradation over twenty-four hours, the maximum weight may be used instead of a 24-hour weight, for example, as measured by taking successive measurements. The weight of the biogel includes the weight of the solution in the biogel.

According to some embodiments, each component has a plurality of similar charges so as to achieve the formation of a physical polymer matrix, e.g., a plurality of functional groups having a negative charge, or a plurality of arms each having a positive charge, or each arm having a functional group of similar charges before crosslinking or other reaction.

Reaction kinetics generally are controlled in light of the particular functional groups unless an external initiator or chain transfer agent is required, in which case triggering the initiator or manipulating the transfer agent may be a controlling step. In some embodiments, the molecular weights of the precursors are used to affect reaction times. In some embodiments, the crosslinking reaction leading to gelation occurs within about 1 second to about 10 minutes or to about 30 minutes; artisans immediately will appreciate that all the ranges and values within the explicitly stated ranges are contemplated, for example, at least 30 seconds, or between 180 seconds to 600 seconds. Gelation time is measured by applying the precursors to a flat surface and determining the time at which there is substantially no flow down the surface when it is titled at an angle of about 60 degrees (i.e., a steep angle, close to perpendicular).

The crosslinking density of the resultant biogel (i.e., the biocompatible crosslinked polymer matrix) is controlled by the overall molecular weight of the anionic and cationic polymer (i.e., hydrophilic polymer) and the number of functional groups available per molecule. For example, a lower molecular weight between crosslinks such as 500 daltons will give much higher crosslinking density as compared to a higher molecular weight such as 10,000 daltons. The crosslinking density also may be controlled by the overall percent solids of the anionic and cationic polymer solutions. Increasing the percent solids increases the probability that an electrophilic functional group will combine with a nucleophilic functional group prior to inactivation by hydrolysis. Yet another method to control crosslink density is by adjusting the stoichiometry of nucleophilic functional groups to electrophilic functional groups. Crosslink density may be affected using ratios of components such as, but not limited to, 1:1; 1:2; 1:3; 1:4; 1:5; 1:6; 1:7; 1:8; 1:9; 1:10; 10:1; 9:1; 8:1; 7:1; 6:1; 5:1; 4:1; 3:1; and 2:1.

The solids content of the biogel may affect its mechanical properties and biocompatibility and reflects a balance between competing requirements. Low solids content may be utilized such as, but not limited to, between about 2.5% to about 25%, including all ranges and values there between, for example, about 2.5% to about 10%, about 5% to about 15%, or less than about 15%. Thus, the solids content that may be utilized include, but are not limited to, 2.5%; 3%; 3.5%; 4%; 4.5%; 5%; 5.5%; 6%; 6.5%; 7%; 7.5%; 8%; 8.5%; 9%; 9.5%; 10%; 10.5%; 11%; 11.5%; 12%; 12.5%; 13%; 13.5%; 14%; 14.5%; 15%; 15.5%; 16%; 16.5%; 17%; 17.5%; 18%; 18.5%; 19%; 19.5%; 20%; 20.5%; 21%; 21.5%; 22%; 22.5%; 23%; 23.5%; 24%; 24.5%; and 25%. Alternately, high solids content also may be utilized.

### 6. Biomedical Devices

According to another embodiment, the biocompatible biogel composition may be disposed on a biomedical device. According to some embodiments, the biomedical device comprises (i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and (ii) an anionic component; and wherein the biogel composition improves at least one anti-adhesive property of the device.

According to another embodiment, the biocompatible biogel composition disposed on a biomedical device further comprises a therapeutic agent. According to some such embodiments, the therapeutic agent is of the form of a microparticle. According to some such embodiments, the therapeutic agent is of the form of a nanoparticle. The therapeutic agent includes, but is not limited to, an analgesic agent, an antimicrobial agent, a steroid agent, a chemotherapeutic agent, a biological agent, a pharmaceutical composition, a growth factor, a cell, or a polypeptide.

The biological agent includes, but is not limited to, a cell, a peptide, a polypeptide, an antibody or an active portion, a fragment, or a derivative thereof.

### 6.1. Coatings of Biomedical Devices

The biocompatible biogel composition also may be used to coat existing medical devices with therapeutic agents. According to another embodiment, the biogel composition may be used to coat existing medical devices to improve their anti-adhesive properties. These biomedical devices include, but are not limited to, orthopedic and cosmetic implants, and cardiovascular grafts and devices, including stents and vascular access grafts.

As used herein, a “biomedical device” refers to a device to be implanted into a subject, for example, a human being, in order to bring about a desired result. Particularly preferred biomedical devices according to this aspect of the invention include, but are not limited to, stents, grafts (i.e., material, especially living tissue or an organ, surgically attached to or inserted into a bodily part to replace a damaged part or compensate for a defect), shunts, stent grafts, fistulas, angioplasty devices, balloon catheters, venous catheters, implantable drug delivery devices, adhesion barriers (including but not limited to carboxymethylcellulose, hyaluronic acid, and PTFE sheets) to separate tissue, wound dressings such as films (e.g., polyurethane films), hydrocolloids (hydrophilic colloidal particles bound to polyurethane foam), hydrogels (cross-linked polymers containing about at least 60% water), other viscous liquids and hydrogel-like species (including but not limited to, those disclosed in US 20030190364), foams (hydrophilic or hydrophobic), calcium alginates (nonwoven composites of fibers from calcium alginate), cellophane, pluronics (ie: poly(ethylene glycol)-block-poly(propylene glycol), and biological polymers.

The term “matrix” as used herein refers to a substance within which something else originates, develops, or is contained.

The term “disposed” as used herein refers to place or put in or on in a sequential, nonsequential, random, nonrandom, uniform, or nonuniform order, density, thickness, concentration, or volume.

The term “disposed on or in” as used herein means that the one or more polypeptides can be either directly or indirectly in contact with an outer surface, an inner surface, or embedded within the biomedical device. “Direct” contact refers to disposition of the polypeptides directly on or in the device, including but not limited to soaking a biomedical device in a solution containing the one or more polypeptides, spin coating or spraying a solution containing the one or more polypeptides onto the device, implanting any device that would deliver the polypeptide, and administering the polypeptide through a catheter directly on to the surface or into any organ.

“Indirect” contact means that the one or more polypeptides do not directly contact the biomedical device. For example, the one or more polypeptides may be disposed in a matrix, such as a gel matrix (such as a heparin coating) or a viscous fluid, which is disposed on the biomedical device. Such matrices can be prepared to, for example, modify the binding and release properties of the one or more polypeptides as required. In one non-limiting example, a heparin coating is disposed on the biomedical device (such as a poly(tetrafluoroethylene) (PTFE) vascular device or sheet) and the one or more polypeptides are disposed on or in a heparin coating; in this example, the one or more polypeptides can be delivered to a subject in need thereof in a controlled manner. In one non-limiting example, the release of the one or more polypeptides from interstitial surfaces of poly(tetrafluoroethylene) (PTFE) vascular devices or sheets can be controlled by first adsorbing or bonding heparin to the surface and/or interstices of the PTFE device followed by adsorption of polypeptide. Alternating layers of heparin and the polypeptide can also be used to increase the polypeptide dose and/or time of release. Under physiological conditions within the body, the kinetics of the association and dissociation of polypeptides disclosed herein to and from heparin will lead to a delayed release profile as compared to release of the polypeptide from a bare PTFE device. In addition, the release profile can be further altered through changes in local temperature, pH or ionic strength. Such controlled release is of great value for use in the various therapeutic treatments for which the biomedical devices can be used, as discussed below.

Heparin coatings on various medical devices are known in the art. Applications in humans include central venous catheters, coronary stents, ventricular assist devices, extracorporeal blood circuits, blood sampling devices, and vascular grafts. Such coatings can be in a gel or non-gel form. As used herein “heparin coating” includes heparin adsorbed to the surface, heparin bonded to the surface, and heparin imbedded in the PTFE polymer surface. An example of a method for bonding the heparin would be to use ammonia plasma to treat, for example, a PTFE surface and reacting the resultant amines with oxidized heparin. Layer-by-layer buildup of the heparin and one or more polypeptides could then be used., to increase polypeptide on the surface and expand the delivery time. Gel forms of the heparin coating can include, but are not limited to, any hydrogel containing heparin either covalently or physically bound to the gel. The heparin coating is disposed on the biomedical device, which includes direct contact with an outer surface or an inner surface of the biomedical device, or embedded within the biomedical device. “Direct” contact refers to disposition directly on or in the device, including but not limited to soaking a biomedical device in a heparin coating solution (wherein the polypeptides may be added as part of the heparin coating solution, or may be subsequently disposed on or in the heparin coating after it is contacted with the device), spin coating or spraying a heparin coating solution onto the device (wherein the polypeptides may be added as part of the heparin coating solution, or may be subsequently disposed on or in the heparin coating after it is contacted with the device), and administering the heparin coating solution containing the polypeptides through a catheter directly on to the surface or into any organ. The physical characteristics and specific composition of the heparin layer can be any that provides the desired release profile of the one or more polypeptides. See, for example, Seal and Panitch, Biomacromoleculcs 2003(4):1572-1582 (2003); US20030190364, incorporated by reference herein in its entirety; and Carmeda BioActive Surface (CBAS™) the product of Carmeda AB in Stockholm, Sweden. “Indirect” contact means that the heparin coating is not directly in contact with the device such as, for example, when an intervening coating is placed between the device surface and the heparin coating. In one non-limiting example, the one or more polypeptides could be initially adsorbed (directly or indirectly), and then adsorbing a heparin coating; this can optionally be followed by subsequent polypeptide layers, heparin layers, or combinations thereof, as desired. As will be understood by those of skill in the art, any sulfated polysaccharide or negatively charged polymer can be used in like manner to heparin as described above, to provide desired release characteristics.

According to another embodiment, a biomedical device comprises one or more isolated polypeptides comprising an amino acid sequence according to general formula I:

Z1-X1-X2-X3-X4 X5-X6-X7-X8-X9-X10-Z2

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein the one or more isolated polypeptides are disposed on or in the device in a biocompatible biogel composition disposed on or in the device.

In another embodiment, a biomedical device comprises one or more isolated polypeptides comprising an amino acid sequence according to general formula I:

Z1-X1-X2-X3-X4 X5-X6-X7-X8-X9-X10-Z2

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent, wherein the one or more isolated polypeptides are disposed on or in the device in a biocompatible biogel composition disposed on or in the device.

In some such embodiments, the polypeptide having an amino acid sequence according to Formula I is of the amino acid sequence KAFAKLAARLYRKALARQLGVAA [SEQ ID NO: 1]. According to another embodiment, the polypeptide having an amino acid sequence according to Formula I is of the amino acid sequence FAKLAARLYRKALARQLGVAA [SEQ ID NO: 2].

According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion.

According to another embodiment, a biomedical device comprises one or more isolated polypeptides comprising an amino acid sequence according to general formula I:

Z1-X1-X2-X3-X4 X5-X6-X7-X8-X9-X10-Z2

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein the one or more isolated polypeptides are disposed in a matrix disposed on the device, wherein the matrix is a heparin coating.

In another embodiment, a biomedical device comprises one or more isolated polypeptides comprising an amino acid sequence according to general formula I:

Z1-X1-X2-X3-X4 X5-X6-X7-X8-X9-X10-Z2

wherein Z1 and Z2 are independently absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent, wherein the one or more isolated polypeptides are disposed in a matrix disposed on the device, wherein the matrix is a heparin coating.

In some such embodiments, the polypeptide having an amino acid sequence according to Formula I is of the amino acid sequence KAFAKLAARLYRKALARQLGVAA [SEQ ID NO: 1]. According to another embodiment, the polypeptide having an amino acid sequence according to Formula I is of the amino acid sequence FAKLAARLYRKALARQLGVAA [SEQ ID NO: 2].

According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion.

According to another aspect, the present invention provides a method to coat a biomedical device with a biocompatible biogel composition, the method comprising steps: (i) providing a biomedical device; (ii) providing a biocompatible biogel composition: (ii) applying the biocompatible biogel composition of step (ii) to the biomedical device of step (i) such that the biocompatible biogel composition forms a homogenous layer on the surface of the biomedical device; thereby providing the biomedical device with a biocompatible biogel composition coating.

According to some embodiments, the step (ii) application is by spraying. According to some embodiments, the step (ii) application is by soaking. According to some embodiments, the step (ii) application is by topical application.

According to another embodiment, the biocompatible biogel composition increases at least one adherence property of the biomedical device.

The term “adherence property” as used herein refers to a characteristic of a substance that is capable of holding materials together in a functional manner by surface attachment that resists separation. Adherence properties may play an important role for in situ biocompatible biogel composition-based therapies. For example, a biocompatible biogel composition that is adherent to a tissue can have good surface-area contact with the surrounding tissue to promote diffusion of drugs or other agents into the tissue. By way of contrast, a failure to adhere will create a diffusion barrier or allow entry of fluids between the drug depot and tissue of the region of interest so that the drugs are washed away. On the other hand, if a biocompatible biogel composition adheres to the tissues around it, or allows tissues to grow and adhere to it, the delivery of the drug may be compromised. Thus, a biocompatible biogel composition depot that adheres tenaciously to the tissue (the biogel's anterior surface) but does not adhere to tissues on its opposing surface (the posterior surface for a coating) or surfaces (for more complex geometries) would be useful. An in-situ formed composition may allow formation of the biogel composition on the region of interest of a tissue with its other surfaces being free or substantially free of tissue contact during the time of gelation and/or crosslinking. In some embodiments, the biogel composition may adhere to specific sites.

A test of adherence of a biogel composition to a tissue may be to apply the biogel composition to the tissue of the region of interest in vitro and show that it is immobilized and not displaced when subjected to external perturbations. By way of contrast, a nonadherent material will be pushed off the tissue.

According to some embodiments, forming a biocompatible biogel composition involves mixing precursors that substantially crosslink after application to a surface, for example, on a tissue of a patient, to form a biodegradable biogel composition depot. Without limiting the invention to a particular theory of operation, it is believed that reactive precursor species that crosslink after contacting a tissue surface will form a three dimensional structure that is mechanically interlocked with the coated tissue. This interlocking contributes to adherence, intimate contact, and essentially continuous coverage of the coated region of the tissue. By way of contrast, conventional materials tend to be nonadhesive to tissue surfaces. For many materials, it is generally unknown whether or not they will be adherent to a tissue, or to any particular tissue.

Another aspect of adherence is that the implant is prevented from moving from the site of its intended use. This tends to increase patient comfort, reduce irritation, and reduce tearing or fluid-flowing reactions that affect the therapeutic agent in the implant. Also, the implant may be placed with precision, for example, between certain tissues or on a tissue, with confidence that it will continue to affect the intended site.

### II. Delivery Systems

The present invention provides a biocompatible biogel composition delivery system for biocompatible biogel compositions that optionally comprise therapeutic agents. According to one aspect, the present invention provides a delivery system that utilizes a biocompatible biogel composition delivery system for injection, deposition or implantation within or upon the body so as to facilitate local therapeutic effects. According to some embodiments, the biocompatible biogel composition delivery system is in the form of a semisolid. According to some embodiments, the biocompatible biogel composition delivery system is in the form of multiparticulates dispersed and suspended in a semisolid. According to some such embodiments, the semisolid comprises a biogel. According to some embodiments, the biocompatible biogel composition delivery system is biodegradable. The term “biodegradable” as used herein refers to material that will degrade actively or passively over time by simple chemical processes, by action of body enzymes or by other similar mechanisms in the human body. The terms “in the body”, “void volume”, “resection pocket”, “excavation”, “injection site”, “deposition site” or “implant site” as used herein are meant to include all tissues of the body without limit, and may refer to spaces formed therein from injections, surgical incisions, tumor or tissue removal, tissue injuries, abscess formation, or any other similar cavity, space, or pocket formed thus by action of clinical assessment, treatment or physiologic response to disease or pathology as non-limiting examples thereof. In one embodiment, the therapeutic agent is a polypeptide having an amino acid sequence of general formula I, or pharmaceutically acceptable salts thereof.

According to another embodiment, the biocompatible biogel composition delivery system comprises in whole or in part a biocompatible, biodegradable, viscous semisolid, wherein the semisolid comprises a biogel.

The delivery of a biocompatible biogel composition results in the formation of a solid or semisolid structure containing a necessary component to produce a gelatinous or jelly-like mass.

In another aspect, the present invention provides a delivery system, which acts as a vehicle for local delivery of therapeutic agents, comprising a lipophilic, hydrophilic or amphophilic, solid or semisolid substance. The therapeutic agent(s) is incorporated and dispersed into the cationic component prior to mixing and formation of the semisolid system. The gelatinous composition is placed within the semisolid delivery apparatus for subsequent placement, or deposition. Being malleable, the gel system easily is delivered and manipulated via the semisolid delivery apparatus in an implant site, where it adheres and conforms to contours of the implantation site, spaces, or other voids in the body as well as completely filling all existing voids. Alternatively, a multiparticulate component, comprised of a biocompatible polymeric or non-polymeric system, is utilized for producing microspheres having a therapeutic agent entrapped therein. Following final processing methods, the microspheres are incorporated into the semisolid system and subsequently placed within the semisolid delivery apparatus so as to be easily delivered therefrom into an implant site or comparable space, whereby the therapeutic agent is subsequently released therefrom by (a) drug release mechanism(s).

According to another embodiment, the biogel composition is used as a drug delivery system where local, controlled release of the therapeutic agent is required. For example, the biogel compositions may be used to deliver highly effective but systemically toxic compounds for (1) oncology applications, (2) pain relief, and/or (3) inflammation reduction. Examples of therapeutic agents useful in such applications include, but are not limited to, analgesic agents, antimicrobial agents, steroid agents, chemotherapeutic agents, or biological agents (including, but not limited to, peptides, proteins, antibodies or active portions, fragments, and derivatives thereof). In some embodiments, the present invention provides a method to reduce pain following surgery.

According to another embodiment, the biogel composition is used as a drug delivery system for delivery of hydrophobic drugs coordinated/organized with cyclodextrin. The term “cyclodextrin” means one of a family of cyclic oligosaccharides comprising 5-8 oligomers of cyclic linked amylose of glucan molecules that forms a hydrophobic interior to accommodate an insoluble compound and a hydrophilic exterior to solubilize in water. According to some embodiments, the drug delivery system comprises: (i) incorporating a cyclodextrin/drug complex into a biogel composition; and (ii) administering the composition of the invention to a patient in need thereof. As used herein the term “hydrophobic” refers to those substances comprising nonpolar molecules that tend to associate with each other in aqueous solution because of the tendency of water molecules to exclude nonpolar molecules.

### III. Methods of Implanting Biocompatible Compositions

Generally, methods for delivery (implantation) include: (i) in situ scaffold formation and (ii) pre-formation. In each case, the therapeutic agent to be delivered is mixed into one component of the rapidly coordinating system. Optionally, the therapeutic is mixed with the anionic component.

1. In situ Formation

The present invention provides a method for in situ formation of a biocompatible biogel implant. One mode of application is to apply a mixture of precursors and other materials (e.g., therapeutic agent, viscosifying agent, accelerator, initiator) through a needle, cannula, catheter, or hollow wire to a region of interest within a subject. The mixture may be delivered, for instance, using a manually controlled syringe or mechanically controlled syringe, e.g., a syringe pump. Alternatively, a dual syringe or multiple-barreled syringe or multi-lumen system may be used to mix the precursors at or near the region of interest.

In one embodiment, a system may involve mixing a drug into a diluent, and drawing an aliquot of the drug/diluent into a 1 ml syringe. A cationic precursor powder is placed into a separate 1 ml syringe. The two syringes are attached via a female-female LUER connector, and the solution moved back and forth between the syringes until the dry precursor is completely dissolved. A solution of multi-armed electrophilic precursor in then water is drawn into a third 1-ml syringe. Using another female-female LUER connector, the user mixes the reconstituted cationic precursor/drug solution with the electrophilic precursor. The solutions rapidly are injected back and forth at least about 10 times to ensure good mixing. The solutions are drawn into 1 syringe and then are available for further use.

Sites where drug delivery depots may be formed include the spine, eye, brain, limb, bone, or any other region of interest within a subject.

The delivery site for placement of a biocompatible biogel composition implant generally is dependent upon the disease or disorder that needs to be treated and the type of drug therapy. For example, steroids, such as dexamethasone and triamicinolone acetonide, may be mixed with the biogel precursors to form a sustained-release drug implant. The liquid biogel then might be injected in situ into the region of interest where it could deliver a constant or tunable release profile of the drug over a over a three to four month time period.

An advantage of a biogel composition implant having three dimensional integrity is that it will tend to resist cellular infiltration and it will be able to prevent the locally administered drug from being phagocytosed and cleared prematurely from the site. Instead, it stays in place until delivered. In contrast, a microparticle, liposome, or pegylated protein tends to be rapidly cleared from the body before it can be bioeffective.

1.1. In situ Barriers

According to another embodiment, the biocompatible biogel compositions may be used to comprise a temporary barrier or gradient device designed to bridge the span between injury and normal healing. These barrier or gradient devices are: (i) suitable for laparoscopic procedures; (ii) conform to the site of application; (iii) allow controlled release of therapeutic agents; and (iv) are customizable to suit specific therapeutic applications.

According to some embodiments, the biocompatible biogel composition provides an adhesion barrier for general surgical procedures. The term “adhesion” as used herein refers to an inflammatory band of scar tissue that binds parts of adjacent tissue, which normally remain separate, together. The scar tissue develops when the body's repair mechanisms respond to any tissue disturbance, including, but not limited to, surgery, infection, trauma, or radiation. Although adhesions can occur anywhere, the most common locations are within the stomach, the pelvis, and the heart. For example, abdominal adhesions are a common complication of surgery, but also occur in subjects who never have had surgery. Adhesions may cause small bowel obstructions in adults and are believed to contribute to the development of chronic pelvic pain. Pelvic adhesions may involve any organ within the pelvis, such as the uterus, ovaries, fallopian tubes, or bladder, and usually occur after surgery. Pelvic inflammatory disease (PID) results from an infection (usually a sexually transmitted disease) that frequently leads to adhesions within the fallopian tubes. Fallopian adhesions may lead to infertility and increased incidence of ectopic pregnancy. Scar tissue also may form within the pericardial sac (meaning the membranes that surround the heart), thus restricting heart function. In addition, infections, such as rheumatic fever, may lead to formation of adhesions on heart valves, leading to decreased heart efficiency. The phrase “reducing scar formation” as used herein refers to any decrease in scar formation that provides a therapeutic or cosmetic benefit. Such a therapeutic or cosmetic benefit may be achieved, for example, by decreasing the size and/or depth of a scar relative to scar formation in the absence of treatment with the methods of the invention, or by reducing the size of an existing scar. As used herein, such scars include adhesion formation between organ surfaces, including, but not limited to, those occurring as a result of surgery.

According to another embodiment, the biocompatible biogel composition increases at least one anti-adhesive property of the device.

According to another aspect, the present invention provides a method for treating an adhesion, the method comprising steps: (a) incorporating a therapeutic agent of interest into a biocompatible biogel composition delivery system, the system comprising (i) providing a first liquid and a second liquid, wherein the first liquid is a coordinating system Component A comprising a hydrophilic polymer with cationic oligomers grafted to the backbone of or within the backbone, and wherein the second liquid is a coordinating system Component B comprising an anionic polymer; (ii) depositing the first liquid and the second liquid into a region of interest through a dual-barrel apparatus whereupon admixing of the first liquid and the second liquid occurs; whereby the first liquid and the second liquid induce rapid formation of a gel or gel-like material; wherein the region of interest is an adhesion; and (b) administering the gel system to a patient in need thereof, thereby reducing the adhesion.

According to some embodiments, the adhesion is an abdominal adhesion. According to some embodiments, the adhesion is a tendon sheath adhesion. According to some embodiments, the adhesion is a cardiac adhesion. According to some embodiments, the adhesion is a meniscal adhesion. According to some embodiments, the surgical procedure is a cosmetic surgical procedure. According to some embodiments, the surgical procedure is a orthopedic surgical procedure. According to some embodiments, the surgical procedure is a surgical repair of a shoulder. According to some such embodiments, According to some embodiments, the adhesion is an idiopathic adhesive capsulitis. According to some embodiments, the surgical procedure is a surgical repair of a knee.

### 1.2. Tissue Fillers

According to another embodiment, the biocompatible gel composition is a tissue filler.

According to another embodiment, the present invention provides a method for filling a dermal or tissue void, the method comprising steps: (i) providing a biocompatible biogel composition, the biogel composition comprising: (a) a cationic component comprising a hydrophilic polymer with cationic oligomers grafted to the backbone or within the backbone, (b) an anionic component; (c) a therapeutic agent; (ii) depositing the biocompatible biogel composition into a region of interest; wherein the region of interest is a dermal or tissue void; thereby reducing the void.

According to some embodiments, the void is a cosmetic void. According to some such embodiments, the void is a void created after breast cancer surgery. According to some such embodiments, the void is a void created after a biopsy. According to some embodiments, the void is a dental void.

### 1.3. Wound Healing

The term “wound” as used herein refers broadly to injuries to the subcutaneous tissue. Such wounds include, but are not limited to fistulas; ulcers; lesions caused by infections; laparotomy wounds; surgical wounds; incisional wounds; and heart tissue fibrosis.

According to another embodiment, the biocompatible biogel composition provides a wound healing matrix to treat a wound. The terms “matrix” and “scaffold” are used interchangeably herein to refer to an artificial structure capable of supporting three-dimensional tissue formation. The matrix may contain therapeutic agents that promote cell attachment and migration, deliver and retain cells and biochemical factors, enable diffusion of vital cell nutrients and expressed products, and exert certain mechanical and biological influences to modify the behavior of the cell phase. According to some embodiments, the scaffold is a biomimetic scaffold (meaning a human-made scaffold that imitates nature). According to some embodiments, the scaffold further comprises derivatives of an extracellular matrix. The extracellular matrix (ECM) refers to an intricate network of macromolecules composed of a variety of proteins and polysaccharides that form the extracellular part of animal tissue that provides, inter alia, structure support to the cells. According to some embodiments, the scaffold is biocompatible. According to some embodiments, the scaffold is biodegradable.

The term “healing” as used herein refers to the process by which the cells in the body regenerate and repair the size of a damaged or necrotic area. Healing may incorporate both the removal of necrotic tissue and the replacement of this tissue. The replacement may occur by (i) regeneration wherein the necrotic cells are replaced by the same tissue as was originally present or (ii) repair wherein injured tissue is replaced by scar tissue. For an injury to be repaired by regeneration, the cell type that was destroyed must be able to replicate. Injury repair occurs when the injury is to cells that are unable to regenerate. Further, damage to the collagen network (e.g. by enzymes or physical destruction), or its total collapse (as can happen in an infarct) cause healing to take place by repair.

Briefly, soon after injury, a wound healing cascade is unleashed. This cascade is usually said to take place in three phases: the inflammatory, proliferative, and maturation stages. In the inflammatory phase, macrophages and other phagocytic cells kill bacteria, debride damaged tissue and release chemical factors such as growth hormones that encourage fibroblasts epithelial cells and endothelial cells which make new capillaries to migrate to the area and divide. In the proliferative phase, immature granulation tissue containing plump active fibroblasts forms. Fibroblasts quickly produce abundant type III collagen, which fills the defect left by an open wound. Granulation tissue moves, as a wave, from the border of the injury towards the center. As granulation tissue matures, the fibroblasts produce less collagen and become more spindly in appearance. They begin to produce the much stronger type I collagen. Some of the fibroblasts mature into myofibroblasts which contain the same type of actin found in smooth muscle, which enables them to contract and reduce the size of the wound. During the maturation phase of wound healing, unnecessary vessels formed in granulation tissue are removed by apoptosis, and type III collagen is largely replaced by type I. Collagen which was originally disorganized is cross-linked and aligned along tension lines. This phase can last a year or longer. Ultimately a scar made of collagen, containing a small number of fibroblasts is left. The process of healing a common incision involves an orchestrated sequence of events in standardized time, beginning with a clot at 0 hours, neutrophil invasion at 3 hours to 24 hours, and mitoses in epithelial bases at 24 hours to 48 hours. According to another embodiment, the wound is a nonhealing wound. The term “nonhealing wound” as used herein refers to wounds that may occur due to tissue hypoxia, i.e., a lack of healing oxygen to the area. Nonhealing wounds include, but are not limited to, a venous ulcer, a diabetic ulcer, and a neural wound. A neural wound may be an injury caused by a stroke, aneurysm, or other trauma or insult. According to some embodiments, the nonhealing wound is a nonhealing burn.

According to another aspect, the present invention provides a method for healing a wound, the method comprising steps: (i) providing a biocompatible biogel composition, the biogel composition comprising (a) a cationic component comprising a hydrophilic polymer with cationic oligomers grafted to the backbone or within the backbone, (b) an anionic component, and (c) a therapeutic agent; (ii) depositing the biocompatible biogel composition into a region of interest within a subject, wherein the region of interest is a wound; wherein the biocompatible biogel composition provides a wound healing matrix thereby treating the wound and facilitating healing.

According to some embodiments, step (ii) depositing is with an apparatus. According to some such embodiments, the apparatus is a dual-barrel apparatus.

According to some such embodiments, the wound healing matrix comprises a therapeutic agent, or a plurality of therapeutic agents.

According to some embodiments, the wound is a non-healing wound. According to some such embodiments, the wound is a wound caused by diabetes. According to some such embodiments, the wound is a wound caused by peripheral vascular disease. According to some embodiments, the wound is an ulcer. According to some such embodiments, the ulcer is a venous ulcer. According to some such embodiments, the ulcer is a diabetic ulcer. According to some such embodiments, the wound is a burn. According to some such embodiments, the burn is a non-healing burn.

### 1.4. Tissue Engineering

According to another embodiment, the biocompatible biogel composition is used for tissue engineering, the biocompatible biogel composition comprising: (a) a biogel for growing the isolated differentiable cells, the biogel comprising (i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and (ii) an anionic component; and (b) isolated differentiable cells, wherein the cells are seeded in the polymer.

The term “graft” as used herein refers to a substance or material that is attached to or incorporated within another substance or material. The term “grafted” as used herein refers to attachment by any mechanism of a substance or material to another substance or material during synthesis or post-synthesis.

According to some embodiments, the isolated differentiable cells are multipotent human mesenchymal cells. According to some such embodiments, the isolated differentiable cells differentiate to chondrocytes. According to some such embodiments, the isolated differentiable cells differentiate to myocytes. According to some such embodiments, the isolated differentiable cells differentiate to osteoblasts.

According to some such embodiments, the biogel forms a scaffold. According to some such embodiments, the scaffold is biocompatible. According to some such embodiments, the scaffold is biodegradable. According to some such embodiments, the scaffold is biomimetic. According to some such embodiments, the scaffold further incorporates therapeutic agents. According to some such embodiments, the scaffold comprises cells. According to some such embodiments, the cells are isolated differentiable cells.

### 1.5. Inflammatory Disorders

According to some embodiments, the biocompatible biogel composition is used to treat inflammation. According to another embodiment, the present invention provides a method for treating inflammation with a biocompatible biogel composition, the method comprising steps: (i) providing a biocompatible biogel composition comprising (a) a cationic component; (b) an anionic component; and (c) a therapeutic agent; (ii) administering the biocompatible biogel composition of step (i) to a region of interest in or on a subject in need thereof, wherein the region of interest contains or is adjacent to an area of inflammation; thereby reducing the inflammation.

According to some embodiments, the therapeutic agents include, but are not limited to, an analgesic agent, an antimicrobial agent, a steroid agent, a chemotherapeutic agent, a biological agent, a pharmaceutical composition, a growth factor, a cell or a polypeptide.

According to some such embodiments, the therapeutic agent is in the form of a microparticle. According to some such embodiments, the therapeutic agent is in the form of a nanoparticle.

According to some such embodiments, the biological agent is a cell, a peptide, a polypeptide, an antibody or an active portion, fragment or derivative thereof.

According to some such embodiments, the biological agent is an isolated polypeptide having an amino acid sequence according to general formula I: Z1-X1-X2-X3-X4 X5-X6-X7-X8-X9-X10-Z2, wherein Z1 and Z2 independently are absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid.

According to some such embodiments, the biological agent is an isolated polypeptide having an amino acid sequence according to general formula I: Z1-X1-X2-X3-X4 X5-X6-X7-X8-X9-X10-Z2, wherein Z1 and Z2 independently are absent or are transduction domains; X1 is selected from the group consisting of A, KA, KKA, KKKA, and RA, or is absent; X2 is selected from the group consisting of G, L, A, V, I, M, Y, W, and F, or is an aliphatic amino acid; X3 is selected from the group consisting of V, L, I, A, G, Q, N, S, T, and C, or is an aliphatic amino acid; X4 is selected from the group consisting of Q, N, H, R and K; X5 is selected from the group consisting of Q and N; X6 is selected from the group consisting of C, A, G, L, V, I, M, Y, W, and F or is an aliphatic amino acid; X7 is selected from the group consisting of S, A, C, T, and G or is an aliphatic amino acid; X8 is selected from the group consisting of V, L, I, and M; X9 is absent or is any amino acid; and X10 is absent or is any amino acid; wherein at least one of the following is true: (a) X3 is N and X7 is not G; (b) X7 is G and X3 is not N; (c) X2 is not L; (d) X4 is not R; (e) X5 is not Q; (f) X6 is not L; (g) X8 is not V; (h) X10 is absent; or (i) X9 and X10 are absent. According to some embodiments, X4 is R, X5 is Q and X8 is V.

According to some such embodiments, the therapeutic agent is an isolated polypeptide having at least 90% amino acid sequence identity to KAFAKLAARLYRKALARQLGVAA [SEQ ID NO: 1], wherein the polypeptide inhibits TNF-α secretion. According to some such embodiments, the therapeutic agent is an isolated polypeptide having at least 90% amino acid sequence identity to FAKLAARLYRKALARQLGVAA [SEQ ID NO: 2], wherein the polypeptide inhibits TNF-α secretion.

According to another embodiment, the inflammatory disorder is selected from the group consisting of hyperplastic scarring, keloids, rheumatoid arthritis, chronic obstructive pulmonary disease, atherosclerosis, intimal hyperplasia, Crohn's disease, inflammatory bowel disease, osteoarthritis, Lupus, tendonitis, psoriasis, gliosis, inflammation, type II diabetes mellitus, type I diabetes mellitus, Alzheimer's disease, and an adhesion. According to another embodiment, the inflammatory disorder comprises glial scarring.

According to some such embodiments, the inflammation is a result of a joint injury. According to some such embodiments, the inflammation is a result of osteoarthritis. According to some such embodiments, the inflammation is a result of rheumatoid arthritis. According to some such embodiments, the step (ii) administration is by surgical implantation. According to some such embodiments, the step (ii) administration is by injection. According to some such embodiments, the step (ii) administration is by topical administration during a surgical procedure.

### 2. Pre-Formation

According to another aspect, the present invention provides a delivery system for a pre-formed biocompatible biogel composition. According to one embodiment, the present invention provides a method for biocompatible biogel delivery, the method comprising the step of implanting a biogel composition.

According to another embodiment, the biocompatible biogel composition is a gel, a slow-release solid or a semisolid compound. According to some embodiments, an adjunct, including, but not limited to, a coating, may be utilized to affect additional properties onto the biogel composition. These additional properties may include, but are not limited to, delayed release, or slowed release of compounds, such as, but not limited to, therapeutic agents. According to another embodiment, the biocompatible biogel composition further comprises a therapeutically effective amount of an active agent and a coating. The active agent may be any of the aforementioned therapeutic agents. The coating can be of any desired material, preferably a polymer or mixture of different polymers. Optionally, the polymer may be utilized during a granulation stage to form a matrix with the active ingredient so as to obtain a desired release pattern of active ingredient. Granules are dosage forms that consist of particles ranging in size from No. 4 (4.76 mm sieve opening) to No. 6 (2.00 mm sieve opening) mesh which are formed when blended powders are moistened and passed through a screen or a special granulator. The gel, slow-release solid or semisolid compound is capable of releasing of the active agent over a desired period of time. The gel, slow-release solid or semisolid compound is implanted in close proximity to a region of interest, whereby the release of the active agent produces a localized pharmacologic effect.

According to another embodiment, the method comprises the step of implanting a biocompatible biogel surgically or injecting a biocompatible gel into the patient to deliver the drug substance at the region of interest. Because the biocompatible biogel, slow-release solid or semisolid agent is delivered specifically (locally) to the region of interest, the dosage required of a therapeutic agent therein will be appropriate to reduce, prevent or circumvent any side effect that might present itself at toxic dosage levels.

The term “implantation” as used herein refers to a procedure whereby a substance or material is inserted or transplaned into a subject.

The biocompatible biogel composition, when it is desirable to deliver it locally, may be formulated for parenteral administration by injection, e.g., by bolus injection. Formulations for injection may be presented in unit dosage form with an added preservative.

The biogel may be used to deliver classes of drugs including steroids, Non-steroidal anti-inflammatory drugs (NSAIDS), intraocular pressure lowering drugs, antibiotics, cytokines, growth factors or others. The biogel may be used to deliver drugs and therapeutic agents, e.g., an anti-inflammatory, a pain reliever, a calcium channel blocker, an antibiotic, a cell cycle inhibitor, or a protein. The rate of release from the biogel will depend on the properties of the drug and the biogel, with factors including drug size, relative hydrophobicities, biogel density, biogel solids content, and the presence of other drug delivery motifs, e.g., microparticles.

The biogel precursors (i.e., the cationic component, the anionic component, and an optional therapeutic agent component) may be used to deliver classes of drugs including steroids, NSAIDS (See Table 1), intraocular pressure lowering drugs, antibiotics, pain relievers, inhibitors or vascular endothelial growth factor (VEGF), chemotherapeutics, anti viral drugs etc. The drugs themselves may be small molecules, proteins, RNA fragments, proteins, glycosaminoglycans, carbohydrates, nucleic acid, inorganic and organic biologically active compounds where specific biologically active agents include but are not limited to: enzymes, antibiotics, antineoplastic agents, local anesthetics, hormones, angiogenic agents, anti-angiogenic agents, growth factors, antibodies, neurotransmitters, psychoactive drugs, anticancer drugs, chemotherapeutic drugs, drugs affecting reproductive organs, genes, and oligonucleotides, or other configurations. The drugs that have low water solubility may be incorporated, e.g., as particulates or as a suspension. Higher water solubility drugs may be loaded within microparticles or liposomes. Microparticles may be formed from, e.g., PLGA or fatty acids.

In some embodiments, the therapeutic agent is mixed with the precursors prior to making the aqueous solution or during the aseptic manufacturing of the functional polymer. This mixture then is mixed with the precursor to produce a crosslinked material in which the biologically active substance is entrapped. Functional polymers may be made from inert polymers like PLURONIC®, TETRONICS® or TWEEN® surfactants to release small molecule hydrophobic drugs.

In some embodiments, the therapeutic agent or agents are present in a separate phase when precursor polymers are reacted to produce a crosslinked polymer network or gel. This phase separation prevents participation of a bioactive substance in the physical crosslinking reaction. The separate phase also helps to modulate the release kinetics of active agent from the crosslinked material or gel, where the term “separate phase” may refer to a biodegradable vehicle, and the like. Biodegradable vehicles in which the active agent may be present include: encapsulation vehicles, such as microparticles, microspheres, microbeads, micropellets, and the like, where the active agent is encapsulated in a bioerodable or biodegradable polymers such as polymers and copolymers of: poly(anhydride), poly(hydroxy acid)s, poly(lactone)s, poly(trimethylene carbonate), poly(glycolic acid), poly(lactic acid), poly(glycolic acid)-co-poly(glycolic acid), poly(orthocarbonate), poly(caprolactone), crosslinked biodegradable biogel networks like fibrin glue or fibrin sealant, caging and entrapping molecules, like cyclodextrin, molecular sieves and the like. In some embodiments, microspheres are made from polymers and copolymers of poly(lactone) s and poly(hydroxy acid).

In using crosslinked materials which are described herein as drug delivery vehicles, the active agent or encapsulated active agent may be present in solution or in suspended form in the cationic component or anionic polymer solution component. The precursor polymers, along with the bioactive agent, with or without an encapsulating vehicle, is administered to the subject along with an equivalent amount of the appropriate aqueous buffers. The physical reaction between the precursor polymer solutions readily takes place to form a crosslinked gel and acts as a depot for release of the active agent to the subject. Such methods of drug delivery find use in both systemic and local administration of an active agent.

A variety of drugs or other therapeutic agents may be delivered using these systems.

In using the crosslinked composition for drug delivery, the amount of precursor components and the dosage agent introduced in the subject necessarily will depend upon the particular drug and the condition to be treated. Administration may be by any convenient means such as syringe, cannula, trocar, catheter and the like.

Certain embodiments of the invention are accomplished by providing compositions and methods to control the release of relatively low molecular weight therapeutic species using biogels. A therapeutic agent first is dispersed or dissolved within one or more relatively hydrophobic rate modifying agents to form a mixture. The mixture may be formed into particles or microparticles, which then are entrapped within a bioabsorbable biogel matrix so as to release the water soluble therapeutic agents in a controlled fashion. Alternatively, the microparticles may be formed in situ during crosslinking of the biogel.

According to another embodiment, the biocompatible biogel composition further comprises biogel microspheres. Biogel microspheres are formed from polymerizable macromers or monomers by dispersion of a polymerizable phase in a second immiscible phase, wherein the polymerizable phase contains at least one component required to initiate polymerization that leads to crosslinking and the immiscible bulk phase contains another component required to initiate crosslinking, along with a phase transfer agent. Pre-formed microparticles containing the water soluble therapeutic agent may be dispersed in the polymerizable phase, or formed in situ, to form an emulsion. Polymerization and crosslinking of the emulsion and the immiscible phase is initiated in a controlled fashion after dispersal of the polymerizable phase into appropriately sized microspheres, thus entrapping the microparticles in the biogel microspheres. Visualization agents may be included, for instance, in the microspheres, microparticles, and/or microdroplets.

Embodiments of the invention include compositions and methods for forming composite biogel-based matrices and microspheres having entrapped therapeutic compounds. In one embodiment, a bioactive agent is entrapped in microparticles having a hydrophobic nature (also termed “hydrophobic microdomains”), to retard leakage of the entrapped agent. In some cases, the composite materials have two phase dispersions, where both phases are absorbable, but are not miscible. For example, the continuous phase may be a hydrophilic network (such as a hydrogel, which may or may not be crosslinked) while the dispersed phase may be hydrophobic (such as an oil, fat, fatty acid, wax, fluorocarbon, or other synthetic or natural water immiscible phase, generically referred to herein as an “oil” or “hydrophobic” phase).

The oil phase entraps the drug and provides a barrier to release by slow partitioning of the drug into the biogel. The biogel phase in turn protects the oil from digestion by enzymes, such as lipases, and from dissolution by naturally occurring lipids and surfactants. The latter are expected to have only limited penetration into the biogel, for example, due to hydrophobicity, molecular weight, conformation, diffusion resistance, and the like. In the case of a hydrophobic drug which has limited solubility in the biogel matrix, the particulate form of the drug may also serve as a release rate modifying agent.

Hydrophobic microdomains, by themselves, may be degraded or quickly cleared when administered in vivo, making it difficult to achieve prolonged release directly using microdroplets or microparticles containing the entrapped agent in vivo. In accordance with the present invention, however, the hydrophobic microdomains are sequestered in a gel matrix. The gel matrix protects the hydrophobic microdomains from rapid clearance, but does not impair the ability of the microdroplets or microparticles to release their contents slowly. Visualization agents may be included, for instance, in the gel matrix or the microdomains.

In one embodiment, a microemulsion of a hydrophobic phase and an aqueous solution of a water soluble molecular compound, such as a protein, peptide or other water soluble chemical is prepared. The emulsion is of the “water-in-oil” type (with oil as the continuous phase) as opposed to an “oil-in-water” system (where water is the continuous phase).

Controlled rates of drug delivery also may be obtained with the system disclosed herein by degradable, covalent attachment of the bioactive molecules to the crosslinked biogel network. The nature of the covalent attachment may be controlled to enable control of the release rate from hours to weeks or longer. By using a composite made from linkages with a range of hydrolysis times, a controlled release profile may be extended for longer durations.

A composition with the precursors mixed therein may be made to have a with viscosity suitable for introduction through a small gauge needle using manual force. A small gauge needle has a diameter less than the diameter of a needle with a gauge of 27, e.g., 28, 29, 30, 31, 32, or 33 gauge, with the gauge being specific for inner and/or outer diameters. Moreover, hollow-tube wires, as used in the intravascular arts, may be used to deliver the materials, including those with inner and/or outer diameters equivalent to the small gauge needles, or smaller. Thus a viscosity of between about 1 centipoise to about 100,000 centipoise may be used; artisans immediately will appreciate that all the ranges and values within the explicitly stated ranges are contemplated e.g., about 10 centipoise to about 10,000 centipoise, less than about 5 centipoise to about 10,000 centipoise, less than about 100 centipoise or about 500 centipoise, or between about 1 centipoise and about 100 centipoise. The viscosity may be controlled, e.g., by choosing appropriate precursors, adjusting solids concentrations, and reaction kinetics. In general, lower concentrations of precursors, increased hydrophilicity and lower molecular weights favor a lower viscosity.

Viscosity enhancers may be used in conjunction with biogel precursors. In general, viscosity enhancers do not react with the biogel precursors to form covalent bonds. While it is appreciated that precursors that are generally free of such bonding may sometimes participate in unwanted side reactions, these have little effect on the biogel so that the precursors are considered “free” of such reactions. For instance, if the precursors react by anionic-cationic reactions, the viscosity enhancers may be free of anions or cations that can form covalent bonds with functional groups of the precursors. Viscosity enhancers are, in general, hydrophilic polymers with a molecular weight of at least 20,000, or from about 10,000 to about 500,000 Daltons; artisans will immediately appreciate that all values and ranges between these explicitly stated values are described, e.g., at least about 100,000 or 200,000. A concentration of about 5% to about 25% w/w may be used, for instance. PEG (e.g., M.W. 100,000 to 250,000) is useful, for example. Viscosity enhancers may be free of anions and/or cations. Viscosity enhancers may be free of one or more functional groups such as hydroxyl, carboxyl, amine, or thiol. Viscosity enhancers may be useful to prevent precursors from running-off a tissue site before the precursor crosslink to form a gel.

According to one aspect, the present invention provides a method for gel delivery for in situ scaffold formation, the method comprising the steps: (1) providing a first liquid and a second liquid, wherein the first liquid is a coordinating system Component A comprising a hydrophilic polymer with cationic oligomers grafted to the backbone or within the backbone, and wherein the second liquid is a coordinating system Component B comprising an anionic polymer; (2) depositing the first liquid and the second liquid into a region of interest through a dual-barrel apparatus whereupon admixing of the first liquid and the second liquid occurs; such that the first liquid and the second liquid induce rapid formation of a gel or gel-like material.

According to one embodiment, the liquid is a solution. The term “solution” as used herein refers to a uniformly dispersed mixture at the molecular or ionic level, of one or more substances (the solute) in one or more other substances (the solvent) and is also used refer to a liquid colloidal dispersion. The term “suspension” as used herein refers to a system in which very small particles (solid, semisolid, or liquid) are more or less uniformly dispersed in a liquid or gaseous medium. If the particles are small enough to pass through filter membranes, the system is a colloidal suspension (or solution).

According to another embodiment, in step (2) the depositing of the first liquid and the second liquid is by injection. According to another embodiment, in step (2) the depositing of the first liquid and the second liquid is via spraying.

According to another embodiment, Component A and Component B are liquids. According to some such embodiments, Component A and Component B are sprayed through small incisions (for example, during laproscopy) into or onto the region of interest. As the liquid droplets coalesce, physical interactions occur, resulting in a gel or a gel-like materials.

According to another embodiment, Component A and Component B are suspensions. According to some such embodiments, Component A and Component B are sprayed through small incisions into or onto the region of interest. As the suspension droplets coalesce, physical interactions occur, resulting in a gel or gel-like material.

According to another embodiment, a therapeutic agent is added to the anionic polymer solution. According to some such embodiments, the anionic polymer solution is a therapeutic suspension. The term “therapeutic suspension” as used herein refers to a suspension, i.e., a mixture in which fine particles are suspended in a fluid, comprising a therapeutic agent or drug.

According to another embodiment, the anionic polymer suspension is drawn into an apparatus; and the cationic solution then is drawn into the same apparatus. Admixing within the apparatus results in physical coordination of a 3-dimensional system. An apparatus may include, but is not limited to, a syringe or amniocentesis needle. Because the system still is able to flow, one may deliver the resulting 3-dimensional system to the site of injury using the apparatus to direct the resulting 3-dimensional system to the site of injury. According to another embodiment, the apparatus is a syringe. According to another embodiment, the end of the apparatus may contain a brush applicator for direct painting of the gel.

According to another aspect, the present invention provides a method for drug delivery, the method comprising steps: (a) incorporating a therapeutic agent of interest into a gel system, the gel system comprising (i) providing a first liquid and a second liquid, wherein the first liquid is a coordinating system Component A comprising a hydrophilic polymer with cationic oligomers grafted to the backbone or within the backbone, and wherein the second liquid is a coordinating system Component B comprising an anionic polymer; (ii) depositing the first liquid and the second liquid into a region of interest through a dual-barrel apparatus where, upon admixing of the first liquid and the second liquid occurs; whereby the first liquid and the second liquid induce rapid formation of a gel or gel-like material; and (b) administering the gel system to a patient in need thereof.

General methods in molecular genetics and genetic engineering useful in the present invention are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise. All technical and scientific terms used herein have the same meaning.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are neither intended to limit the scope of what the inventors regard as their invention nor they intended to represent that the experiments below are all or the only experiments performed.

### Example 1

### Biocompatible Biogel Composition: Polyvinyl Amine

In one embodiment, the cationic component of the biocompatible biogel composition is poly(N-vinyl formamide) hydrolyzed to produce polyvinylamine covalently coupled to a multivalent hydrophilic polymer or co-polymer backbone, such as poly(ethylene glycol) or dextran. Instructions and protocols for synthesis routes are given in several published papers including a) Gu, Zhu, and Hrymak, 2002, J Appl Poly Sci, 86: 3412-3419, b) Tanaka and Senju, 1976, Bulletin of the Chemical Society of Japan, 49(10): 2821-2823, c) Fisher and Heitz, 1994, Macromol Chem Phys, 195: 679-687, and d) Achari and Coqueret, 1997, J Polym Sci A: Polym Chem, 35: 2513-2520. The degree of the polymerization of the polyvinylamine is less than or equal to 100%. In some embodiments, the anionic component is heparin, heparan sulfate, dermatan sulfate, chondroitin sulfate, hyaluronic acid, or dextran sulfate.

### Example 2

### Biocompatible Biogel Composition: Small Molecule Mimicking Cationic Peptides

In one embodiment, the cationic component of the biocompatible biogel composition are small molecules mimicking cationic peptides, such as those described by Choi et al. (Choi, Clements, et al., 2005, Angewandte Chemie, 44(41): 6685-6689) that are covalently coupled to a multivalent hydrophilic polymer or co-polymer backbone, such as poly(ethylene glycol) or dextran. The anionic component is heparin, heparan sulfate, dermatan sulfate, chondroitin sulfate, hyaluronic acid, or dextran sulfate.

### Example 3

### Biocompatible Biogel Composition: Guanidinyl Groups

The cationic component of the biocompatible biogel composition is polymers or co-polymers containing guanidinyl groups that are covalently linked to a multivalent hydrophilic polymer or co-polymer backbone, such as poly(ethylene glycol) or dextran. Agmatine is functionalized with acryloyl chloride to create a guanidinyl acrylamide. The guandinyl acrylamide is polymerized by itself or in the presence of other monomers (such as allyl amine, N-2 aminoethyl methacrylamide, or N-3-aminopropyl methacrylamide) to create polymers or co-polymers with total degree of polymerization less than or equal to 100%. In some embodiments, the anionic component is heparin, heparan sulfate, dermatan sulfate, chondroitin sulfate, hyaluronic acid, or dextran sulfate.

### Example 4

### Biocompatible Biogel Composition

In some embodiments, the cationic component of the biocompatible biogel composition is polymers or co-polymers of monomers containing primary amines, secondary amines, or tertiary amines that are covalently coupled to a multivalent hydrophilic polymer or co-polymer backbone, such as poly(ethylene glycol) or dextran. Allyl amine is polymerized by itself or copolymerized with N-2-N,N-dimenthylamino ethly methacrylamide to create polymers or co-polymers with a total degree of polymerization less than or equal to 100%. In some embodiments, the anionic component is heparin, heparan sulfate, dermatan sulfate, chondroitin sulfate, hyaluronic acid, or dextran sulfate.

### Example 5

### Biocompatible Biogel Composition

In some embodiments, the cationic component of the biocompatible biogel composition is the polymers described in Example 4 that are copolymerized with hydrophilic or hydrophobic monomers and covalently coupled to a multivalent hydrophilic polymer or co-polymer backbone, such as poly(ethylene glycol) or dextran. Allyl amine is co-polymerized with styrene and/or N-isopropyl acrylamide to create a co-polymer with a total degree of polymerization less than or equal to 100%. In some embodiments, the anionic component is heparin, heparan sulfate, dermatan sulfate, chondroitin sulfate, hyaluronic acid, or dextran sulfate.

### Example 6

### Delivery of Mesenchymal Stem Cells to Repair Damaged Cartilage

Bone marrow aspirates of 30-50 mL will be obtained from healthy human donors. Marrow samples will be washed with saline and centrifuged over a density cushion of ficoll. The interface layer will be removed, washed, and the cells counted. Nucleated cells recovered from the density separation will be washed and plated in tissue culture flasks in Dulbecco's Modified Eagle's Medium (DMEM) containing 10% fetal bovine serum (“FBS”, HyClone Laboratories, Inc.). Non-adherent cells will be washed from the culture during biweekly feedings. Colony formation will be monitored for a 14-17 day period. MSC's will be passaged when the tissue culture flasks are near confluent. At the end of the first passage, MSCs will be enzymatically removed from the culture flask using trypsin-EDTA and replated at a lower density for further expansion. At the end of the second passage, MSC's will be either entrapped within the gel/gel-like system of the present invention or cryopreserved until future use.

The hMSC cells will be identified as multipotent stem cells based on surface marker characterization, which distinguishes the stem cells from other cell types in the bone marrow, for example white blood cells. Cells expressing CD44 (CD44⁺) and the absence of CD45 (CD45⁻) and CD34 (CD34⁻) surface antigens will be verified by fluorescence-activated-cell-sorter.

Mesenchymal stem cells will be entrapped within the biocompatible biogel composition as follows. The mesenchymal stem cells will be suspended within the anionic component, and the two components will be delivered in situ through a duel barrel syringe into the sight of a torn meniscus. The biogel material will act as a scaffold to support mesenchymal stem cell differentiation and to support repair of the damaged cartilage.

### Example 7

### Delivery of Stem Cells to Repair Cardiovascular or Cerebrovascular Injury

Acute myocardial infarction model.

Rats will be anesthetized using 100 mg/kg ketamine and 5 mg/ml diazepam. Mechanical ventilation will be used. A 1.5 cm laterial thoracotomy of the 5th interrib space will be performed to expose the heart. The left aortic descending artery will be ligated using a 7.0 suture to create a myocardial infarction {Tran, 2007 #1578}. The incisions will be closed and animals allowed to recover for 4 months.

After this 4 month period, mesenchymal stem cells or haematopoietic stem cells (4×10⁹ cells/ml) will be suspended in a 10% (w/v) gel or gel-like material by combining cells with the anionic component before mixing or by premixing the biogel material prior to adding the cells suspension. Cells will be incorporated by gentle trituration prior to injection into the infarcted tissue. 50 μl of the suspension will be injected into the middle of the infarcted tissue.

### Example 8

### Treatment of Arthritic Joints

Microparticles of poly(lactide-co-glycolide) encapsulating a therapeutic agent, including, but not limited to, a nonsteroidal anti-inflammatory (NSAID), are delivered to an arthritic joint using the dual-barrel syringe method. A 20 (wt %) solution of the cationic graft-co-polymer is added to one barrel of a dual barrel syringe and a 1 (wt %) solution of the anionic polymer is added to the other barrel of a dual barrel syringe. The biocompatible biogel composition is formed upon mixing of the contents of the two barrels of the dual barrel syringe, and the gel/gel-like system is delivered laparoscopically to an area of interest on or within a patient in need thereof.

The microparticles, which are entrapped within the biocompatible biogel composition, release the therapeutic agent locally to affect the arthritic joint.

### Example 9

### Cancer Therapy

The biocompatible biogel composition of the present invention may be used as a cancer therapeutic. In one example, a 10% (w/v) gel/gel-like composition is premixed or mixed upon delivery and is formed by adding the anionic component to the cationic component containing a chemotherapeutic agent, such as paclitaxel or doxorubicin. The system is delivered via a syringe or dual barrel syringe and injected either directly into a tumor or adjacent to a tumor mass.

### Example 10

### Coating of Biomedical Devices

The biocompatible biogel composition may be applied to medical devices, including, but not limited to, stents, catheters, vascular or prosthetic grafts, sutures, orthopedic implants, and bone screws. In one example, the gel/gel-like system is applied to a medical device in a manner similar to that of the Carmeda Bio-Active Surface (CBAS™). Anionic polymer is covalently attached to the surface of a medical device. Then, the medical device is coated with a layer of hydrophilic polymer, which associates with the covalently attached anionic polymer. Another layer of anionic polymer is added to the cationic layer, and a cationic layer is added to the resulting anionic layer. This coating process continues until 5-20 anionic/cationic layers are added to the medical device. The biocompatible biogel composition may contain a pharmaceutical, drug, therapeutic, cell, protein, or other molecule intended to result in an intended biological response or effect. The pharmaceutical, drug, therapeutic, cell, protein or other molecule is added to either the anionic or cationic solutions or to both the anionic or cationic solutions used to prepare the biocompatible biogel.

### Example 11

### Scaffolds for Tissue Engineering

The biocompatible biogel composition of the present invention may be used for growth of artificial bone or cartilage. The biocompatible biogel composition may incorporate controlled or diffusion based release of growth factors or drugs to help maintain an osteoblast, osteocyte, and/or chondrocyte phenotype, to recruit mesenchymal or other progenitor cell types, or to allow for improved bone or cartilage extracellular matrix production. Further, biocompatible biogel composition may incorporate autologous, allogenic, or zenogenic cells.

In one example, chondrocytes are isolated from knee joints by obtaining thin slices of articular cartilage using a scalpel. The cartilage will be rinsed with phosphate buffered saline and digested at 37° C. for 5 hours using a solution of Dulbecco's Modified Eagle Medium (“DMEM”) containing 10 mM HEPES, pH 7.4, 2% serum, 2 mM glutamine, penicillin/streptomycin antibiotics and 125 units/ml collagenase [Fragonas E, Valente M, Pozzi-Mucelli M, Toffanin R, Rizzo R, Silvestri F, Vittur F, Aricular cartilage repair in rabbits by using suspensions of allogenic chondrocytes in alginate, Biomaterials, 2000, 21(8):795-801] Chondrocytes will be seeded in monolayer at a density of 20,000 cell/cm² and expanded in culture [Marijnissen WJCM, et al. Tissue-engineered cartilage using serially passaged articular chondrocytes. Chondrocytes in alginate, combined in vivo with a synthetic (E210) or biologic degradable carrier (DBM), Biomaterials, 2000, 21(6):571-580]. Chondrocytes at a density of 30×10⁶ cells/ml will be suspended in a 10% (w/v) gel or gel-like material by combining cells with the anionic component before mixing or by premixing the gel/gel-like material prior to adding the cell suspension. Cells will be incorporated by gentle trituration prior to injection into an articular cartilage defect (50-1000 μl); the system can be further protected from displacement by a periosteal flap.

The biocompatible biogel composition also can be used as an in vitro matrix for tissue engineering. Chondrocytes at a density of 30×10⁶ cell/ml will be suspended in a 10% (w/v) gel or gel-like material by combining cells with the anionic component before mixing or by premixing the gel/gel-like material prior to adding the cell suspension. The cell-containing biocompatible biogel composition then will be cultured for two or more weeks in a humidified incubator at 37° C. to allow chondrocytes to synthesize a cartilage matrix. The synthesized cartilage matrix then can be implanted surgically into a defect site.

### Example 12

### Stabilization of Neural Injury

The biocompatible biogel composition of the present invention may be used to stimulate or maintain “devolvement” of the extracellular matrix in instances of neural injury to allow nerve regeneration. Optionally, it may incorporate slow-release chondroitinase or hyaluronidase to keep “adult-like” matrix constituents from stabilizing the injury, thus allowing tissue regeneration.

Sprague Dawley rats (200-300 gm) will be subjected to spinal cord injury using transection. Following halothane anesthesia, dorsal laminectomies at T9 will expose the cord. Complete transection leaving a 2 mm gap will be achieved using an iris scalpel. Installation of the gel/gel like system or saline (control) into the injured cord will be achieved using 50-100 μl injections. A 20 (wt %) solution of the cationic graft-co-polymer with or without incorporation of a slow release agent, will be added to one barrel of a dual barrel syringe and a 1 (wt %) solution of the anionic polymer will be added to the other barrel of a dual barrel syringe. Closure will be accomplished using absorbable suture material. The animals will be allowed to recover on warmed blankets. Prophylactic antibiotics will be administered for one week, and subsequently if needed. Urinary bladders will be emptied thrice daily by mechanical expression for the first week, and twice daily thereafter to prevent urinary tract infections. Animals will be sacrificed at two time points to provide assessment of the onset and sustained regeneration of axons (typically in cohorts of 6 and 16) on days 7 and 84 respectively. The day 7 time points should allow a determination of whether there is an excessive proliferation of astrocytes and whether there is a chronic immune response. Day 84 will provide information on axonal regeneration (Coumans, J. V., T. T.-S. Lin, et al. (2001). “Axonal regeneration and functional recovery after complete spinal cord transection in rats by delayed treatment with transplants and neurotrophins.” Journal of Neuroscience 21(23): 9334-9344). A larger number of animals is needed for day 84 animals so that longitudinal and axonal sectioning as well as neuroanatomical tracing may be performed (Woerly, S., V. D. Doan, et al. (2001). “Spinal cord reconstruction using Neurogel™ Implants and functional recovery after chronic injury.” Journal of Neuroscience Research 66: 1187-1197).

### Example 13

### Tissue Filler

The biocompatible biogel composition of the present invention may be used as a soft-tissue filler substance. In one example, the gel/gel-like system is used as a facial filler. A 20% (w/v) gel or gel-like material is formed by combining the anionic component with the cationic component upon injection or by premixing. The biocompatible biogel composition is injected into glabellar wrinkles, nasolabial folds, lips, nose, or infraorbital regions using volumes ranging from 0.5 to 2.0 ml. Before injection, regional or nerve block anesthesia (e.g., 2% lidocaine) is used. Then, a 25 or 27 gauge needle is used to inject the gel/gel-like system into the dermis [Jacovella P F, Long-lasting results with hydroxylapatide (Radiesse) facial filler, Plastic and Reconstructive Surgery, 2006, 118(3S):15S-21S]. The biocompatible biogel composition is used alone or be mixed with pharmaceuticals, cells, or other molecules to facilitate autologous extracellular matrix production or other biological functions that would augment tissue volume.

### Example 14

### Inhibition of Abdominal Adhesions

Anesthetized rats will be prepped for surgery by shaving the lower abdomen and cleaning it with iodine. Animals will undergo a midline celiotomy, the cecum will be identified and placed onto a gauze pad and saline used to keep the tissue moist. The cecum wall will be abraded using a 1×1 cm electrosurgical tip cleaner until bleeding is noted on the anterior surface. A 1.6×0.8 mm defect will be created in the peritoneum and underlying muscle using a 0.8 mm biopsy punch. The abdominal cavity will be irrigated prior to application of treatments. The A 20 (wt %) solution of the cationic graft-co-polymer will be added to one barrel of a dual barrel syringe and a 1 (wt %) solution of the anionic polymer with or without drug will be added to the other barrel of a dual barrel syringe. The biocompatible biogel composition will form upon mixing of the contents of the two barrels of the dual barrel syringe, and the gel/gel-like system will be delivered directly to the injured tissue. Due to the rapid gelling nature and low modulus of the barrier, it will be injected into place to allow it to conform to the damaged tissue and not flow throughout the abdominal cavity. Care will be taken to ensure that the barrier does separate the damaged tissues [Buckenmaier, C. C., 3rd, et al., Comparison of antiadhesive treatments using an objective rat model. Am Surg, 1999. 65(3): p. 274-82]. [Zong, X., et al., Prevention of postsurgery-induced abdominal adhesions by electrospun bioabsorbable nanofibrous poly(lactide-co-glycolide)-based membranes. Ann Surg, 2004. 240(5): p. 910-5]. Fourteen days post-surgery, the rats again will be anesthetized as described above and a surgeon who is blinded to the treatments will perform a second celiotomy to evaluate the extent and severity of the adhesions. The vast majority of abdominal adhesion studies use a visual analogue scoring system rather than histology. The following scoring system therefore will be used: 0=no adhesions, 1=thin and filmy, easily separated adhesions, 2=significant and filmy, difficult to separate tissue and 3=severe with fibrosis, instruments required to separate tissue. The number of animals within each group with adhesions and the severity of adhesions will be noted and then compared across groups using ANOVA analysis to determine the best treatment combination (barrier, rate of release and drug concentration) to inhibit adhesions.

### Example 15

### Effect of Heparin-binding Peptides on Cellular Viability

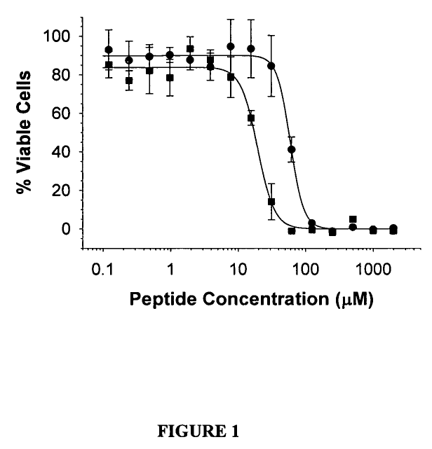

In one example to show toxicity of two heparin binding peptides, 3T3 fibroblasts were treated with various concentrations of two different heparin binding peptides. Cell viability was assessed using the CellTiter 96® AQueous reagent (Promega, Madison, Wis.) after 1 hour at 37° C. in a humidified incubator with 5% CO₂, following the manufacturer's protocol, incorporated by reference herein. Briefly, 3T3 fibroblasts were cultured at 37° C. in a humidified incubator with 5% CO₂ using Dulbecco's modified Eagle Medium (DMEM) containing 10% fetal bovine serume (FBS), pen/strep, and 2 mM glutamine (Invitrogen). Serial dilutions of dG-PGB1 and W-PBD-1 using PBS as the diluent, and 75 μl of the dilutions were transferred in triplicate into the wells of a 96-well microtiter plate. Next, 75,000 cells, recently trypsinized with 0.25% trypsin with EDTA (Invitrogen) and resuspended in PBS to a concentration of 1,000,000 cells/ml, were added to each well on the microplate. The plate was incubated at 37° C. and 5% CO₂ for 1 hour, and then centrifuged for 5 minutes at 20° C. at 1500 rpm. After removing the supernatant from each well, 50 μl trypsin was added to digest any remaining soluble heparin binding peptide or peptide bound to the cell surface, and the plate was incubated for 5 minutes at 37° C. and 5% CO₂. Next, 50 μl DMEM with 10% FBS was added to each well to neutralize trypsin, and the plates were centrifuged for 5 minutes at 20° C. at 1500 rpm. Following supernatant removal, the cell pellets were washed twice with DMEM containing 10% FBS. Then, 100 μl serum containing media was added followed by 20 μl CellTiter 96® Aqueous reagent (Promega, Madison, Wis.). The plates were incubated at 37° C. in a humidified atmosphere with 5% CO₂ for 3 hours to allow the metabolically sensitive substrate to develop. Finally, the absorbance at 490 mm was measured with a microplate spectrophotometer. Controls also were tested in triplicate and consisted of 75,000 cells treated only with PBS and 75,000 cells treated with PBS containing a final concentration of 0.1% (v/v) Triton X-100. The amount of viable cells was determined by subtracting the absorbance readings of media containing blank wells from every other well and comparing the ratio of the absorbance reading for each sample with that of the positive control. FIG. 1 shows a plot of viable cells (%) versus peptide concentration (μM). The cellular viability was adversely affected by two heparin binding peptides, dansyl-GKAFAKLAARLYRKAGC (dG-PBD1) (19.3±1.1 μM) and WKAFAKLAARLYRKAGC (W-PBD1) [SEQ ID NO: 1] (58.1±7.3 μM).

### Example 16

### Synthesis of Polymers

### Example 16.1

### Synthesis of Base Polymer

Base polymer components of the biocompatible biogel composition comprising 50% acrylamide, 35% acrylic acid and 15% styrene were synthesized. Briefly, dioxane (100 ml) (EMD Chemicals, San Diego, Calif.), acrylamide (3 g) (Invitrogen, Carlsbad, Calif.), styrene (1.39 g) (99% purity; Alfa Aesar, Ward Hill, Mass.) and acrylic acid (2.04 mL) (99% purity; Alfa Aesar, Ward Hill, Mass.) were added to a 3-neck round bottom flask under N₂ at 25° C., then heated to 60° C. in an oil bath.

The polymerization initiator 2,2′-azobisisobutyronitrile (“AIBN”) (0.139 g) (98% purity; Sigma Aldrich, St. Louis, Mo.), predissolved in dioxane (10 ml), then was added and the reaction allowed to continue for 24 hours with constant stirring. The product (polymer “A”) was precipitated in ethyl ether (Mallinckrodt Chemicals), then dried in a vacuum oven for 3 days.

### Example 16.2

### Functionalization of Base Polymer with N-hydroxysuccimide (NHS)

The base polymers was functionalized with N-hydroxysuccimide (“NHS”). Briefly, anhyrdous methylene chloride was formed by drying dichloromethane (“DCM”) (400 ml) (Mallinckrodt Chemicals, Hazelwood, Mo.) through magnesium sulfate (Mallinckrodt Chemicals, Hazelwood, Mo.). The anhydrous methylene chloride (200 ml) then was added with base polymer (1 g) in a 3-neck round bottom flask under N₂ at 25° C.

The acrylic acid of the each base polymer was further functionalized. NHS (4.001 g) (98% purity; Sigma Aldrich) and N,N′-diisopropylcarbodiimide (“DIC”) (5.33 mL) (99% purity; Sigma Aldrich) were added to base polymer A in excess of 8:3 based on the molar yield of acrylic acid.

The reaction was allowed to continue for 24 hours with constant stirring. NHS-polymer A was precipitated in ethyl ether (Mallinckrodt Chemicals), then dried in a vacuum oven for 3 days.

### Example 16.3

### Functionalization of NHS-Polymer with Agmatine

The NHS-polymer was further functionalized with agmatine. Briefly, sodium biocarbonate (0.1N; 25 mL) (Sigma Aldrich) and an excess of agmatine sulfate (2 M; Biosynth Chemistry & Biology, Itasca, Ill.) were added to a 1-neck round bottom flask at 25° C.

The amount of the excess agmatine sulfate to be added is calculated based on 1 g of the NHS-polymer that the solution is to be subsequently added to. Thus, for the sodium bicarbonate solution destined for addition of NHS-polymer A, 1.370 g of agmatine sulfate was added.

NHS-polymer A (1 g) was dissolved in N,N-dimethylformamide (“DMF”) (10 mL) and added drop-wise to the appropriate sodium biocarbonate/agmatine sulfate solution in an ice bath. The reaction was continued for 45 hours with constant stirring at 25° C., diluted with deionized water (25 mL), then dialyzed against deionized water for 4 days within a 500 dalton molecular weight cutoff cellulose membrane (Spectrum Laboratories, Rancho Dominguez, Calif.), with the dialyzing water changed thrice daily. The resulting NHS-agmatine-polymer A product was frozen (−80° C.), then lyophilized for 3 days.

### Example 16.4

### Binding of NHS-Agmatine-Polymer

The binding efficiency and conductivity of the functionalized base polymer NHS-agmatine-polymer A obtained in Example 16.3 was studied. Briefly, using an AKTA Explorer fast protein liquid chromatograph (Amersham Biosciences-GE Healthcare, Piscataway, N.J.), NHS-agmatine polymer A was loaded onto a 1.0 mL HisTrap™ HP heparin chromatography column (GE Healthcare, Milwaukee, Wis.). The polymer was eluted using a 20 mM sodium phosphate/1 M sodium chloride buffer (elution buffer). NHS-agmatine-polymer B was loaded and eluted in an identical manner. The conductivity at peak elution was measured and normalized relative to 100% elution buffer conductivity. Table 1 shows the conductivity of the polymer and the corresponding concentration of sodium chloride required for elution.

TABLE 1

| | Beginning of Peak | | Center of Peak | | End of Peak | |
|---|---|---|---|---|---|---|
| | | Sodium | | Sodium | | Sodium |
| | | chloride | | chloride | | chloride |
| | | concentration | | concentration | | concentration |
| | Cond | at elution | Cond | at elution | Cond | at elution |
| | (mS/cm) | (mM) | (mS/cm) | (mM) | (mS/cm) | (mM) |
| Polymer | 38.7 | 480 | 50.9 | 630 | 63.7 | 800 |
| A | | | | | | |

NHS-agmatine-polymer A begins to elute at a conductivity of 38.7 mS/cm and continues to elute through greater than 63.7 mS/cm. The binding buffer (20 mM sodium phosphate, pH 7.4) had a conductivity of 0.8 mS/cm, and the elution buffer (20 mM sodium phosphate, pH 7.4 with 1 M sodium chloride) had a conductivity of 80 mS/cm. Therefore, because of the linear relationship between conductivity and sodium chloride concentration, the conductivities recorded for polymer A indicate that the polymer began to elute at a sodium chloride concentration of 480 mM and continued to elute through 800 mM sodium chloride.

The observed conductivity of NHS-agmatine-polymer A is consistent with that observed of polymer compositions that polymerize to biogels in physiological settings.

We claim:

1. A method for treating a wound with a biocompatible biogel composition, the method comprising the steps:
(a) providing a biocompatible biogel comprising
(i) a cationic component, wherein the cationic component comprises a hydrophilic polymer having a molecular weight greater than about 3000 g/mole, but less than about 10,000,000 g/mole, to which at least about 3 cationic oligomers, but no more than 1,000,000 cationic oligomers is grafted; and
(ii) an anionic component; and
(iii) optionally a therapeutically effective amount of a therapeutic agent; and
(b) forming a biocompatible matrix to support wound healing.

2. The method according to claim 1 wherein the therapeutic agent is selected from the group consisting of an analgesic agent, an antimicrobial agent, a steroid agent, a chemotherapeutic agent, a biological agent, a pharmaceutical composition, a growth factor, a cell, or a polypeptide.

3. The method according to claim 2, wherein the therapeutic agent is a microparticle form.

4. The method according to claim 2, wherein the therapeutic agent is a nanoparticle form.

5. The method according to claim 2, wherein the biological agent is an isolated cell.

6. The method according to claim 2, wherein the biological agent is an isolated peptide, an isolated polypeptide, an isolated antibody or an isolated active portion, fragment or derivative thereof.

7. The method according to claim 2, wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid.

8. The method according to claim 2, wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid;
wherein at least one of the following is true:
(a) X3 is N and X7 is not G;
(b) X7 is G and X3 is not N;
(c) X2 is not L;
(d) X4 is not R;
(e) X5 is not Q;
(f) X6 is not L;
(g) X8 is not V;
(h) X10 is absent; or
(i) X9 and X10 are absent.

9. The method according to claim 8, wherein X4 is R, X5 is Q and X8 is V.

10. The method according to claim 1, wherein the therapeutic agent is an isolated polypeptide having at least 90% amino acid sequence identity to FAKLAARLYRKALARQLGVAA [SEQ ID NO: 2], wherein the polypeptide inhibits TNF-α secretion.

11. The method according to claim 1, wherein the therapeutic agent is an isolated polypeptide having at least 90% amino acid sequence identity to KAFAKLAARLYRKALARQLGVAA [SEQ ID NO: 1], wherein the polypeptide inhibits TNF-α secretion.

12. The method according to claim 1, wherein the hydrophilic polymer comprises acrylamide, styrene, acrylic acid and a polymerization initiator.

13. The method according to claim 1, wherein the hydrophilic polymer comprises (i) 50% acrylamide, (ii) 15% styrene, (iii) 35% acrylic acid and (iv) 2,2′-azobisisobutyronitrile 1/100 molar ratio to monomers.

14. The method according to claim 1, wherein the hydrophilic polymer comprises (i) 50% acrylamide, (ii) 15% styrene, (iii) 35% acrylic acid and (iv) 2,2′-azobisisobutyronitrile 1/200 molar ratio to monomers.

15. The method according to claim 12, wherein the acrylic acid is functionalized with a guanidyl group.

16. The method of claim 15, wherein the guanidyl group is agmatine sulfate.

17. The method of claim 15, wherein the guanidyl group is of arginine, or a derivative thereof.

18. The method according to claim 1, wherein the wound is a nonhealing wound.

19. The method according to claim 18, wherein the nonhealing wound is a venous ulcer.

20. The method according to claim 18, wherein the nonhealing wound is a diabetic ulcer.

21. The method according to claim 18, wherein the nonhealing wound is a nonhealing burn.

22. The method according to claim 1, wherein the wound is a neural wound.

23. A method for supporting differentiation of isolated differentiable cells into a mature phenotype, the method comprising steps:
(1) providing a biocompatible biogel composition comprising:
(a) a biogel for growing isolated differentiable cells, the biogel comprising
(i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and
(ii) an anionic component; and
(b) isolated differentiable cells;
(2) administering the biocompatible biogel composition into a region of interest to a subject in need thereof;
(3) forming a tissue scaffold to support differentiation of isolated cells into a mature phenotype.

24. The method according to claim 23, wherein the isolated differentiable cells are multipotent human mesenchymal cells.

25. The method according to claim 23, wherein the biogel supports differentiation of the isolated differentiable cells into a mature phenotype, and wherein the mature phenotype is a chondrocyte.

26. The method according to claim 23, wherein the biogel supports differentiation of the isolated differentiable cells into a mature phenotype, wherein the mature phenotype is a myocyte.

27. The method according to claim 23, wherein the biogel supports differentiation of the isolated differentiable cells into a mature phenotype, wherein the mature phenotype is an osteoblast.

28. The method according to claim 23, wherein the region of interest is in or adjacent to a bone tissue.

29. The method according to claim 23, wherein the region of interest is in or adjacent to a cardiac tissue.

30. The method according to claim 23, wherein the region of interest is in or adjacent to a neural tissue.

31. The method according to claim 23, wherein the region of interest is in or adjacent to a wound.

32. The method according to claim 23, wherein the region of interest is in or adjacent to a nonhealing wound.

33. The method according to claim 23, wherein the hydrophilic polymer comprises acrylamide, styrene, acrylic acid and a polymerization initiator.

34. The method according to claim 23, wherein the hydrophilic polymer comprises (i) 50% acrylamide, (ii) 15% styrene, (iii) 35% acrylic acid and (iv) 2,2′-azobisisobutyronitrile 1/100 molar ratio to monomers.

35. The method according to claim 23, wherein the hydrophilic polymer comprises (i) 50% acrylamide, (ii) 15% styrene, (iii) 35% acrylic acid and (iv) 2,2′-azobisisobutyronitrile 1/200 molar ratio to monomers.

36. The method according to claim 33, wherein the acrylic acid is functionalized with a guanidyl group.

37. The method according to claim 36, wherein the guanidyl group is agmatine sulfate.

38. The method according to claim 36, wherein the guanidyl group is of arginine, or a derivative thereof.

39. A biomedical device comprising a biocompatible biogel composition disposed on the device, the biogel composition comprising
(i) a cationic component, wherein the cationic component comprises a hydrophilic polymer having a molecular weight from about 3000 g/mole to about 10,000,000 g/mole, wherein the hydrophilic polymer comprises at least about 3 cationic oligomer grafts to about 1,000,000 cationic oligomer grafts; and
(ii) an anionic component; and
wherein the biogel composition improves at least one anti-adhesive property of the device.

40. The biomedical device according to claim 39, wherein the biocompatible biogel composition further comprises a therapeutic agent.

41. The biomedical device according to claim 40, wherein the therapeutic agent is a microparticle form.

42. The biomedical device according to claim 40, wherein the therapeutic agent is a nanoparticle form.

43. The biomedical device according to claim 40, wherein the therapeutic agent is selected from the group consisting of an analgesic agent, an antimicrobial agent, a steroid agent, a chemotherapeutic agent, a biological agent, a pharmaceutical composition, a growth factor, a cell, or a polypeptide.

44. The biomedical device according to claim 43, wherein the biological agent is an isolated cell.

45. The biomedical device according to claim 43, wherein the biological agent is an isolated peptide, an isolated polypeptide, an isolated antibody or an isolated active portion, a fragment, or a derivative thereof.

46. The biomedical device according to claim 43, wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid.

47. The biomedical device according to claim 43 wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid;
wherein at least one of the following is true:
(a) X3 is N and X7 is not G;
(b) X7 is G and X3 is not N;
(c) X2 is not L;
(d) X4 is not R;
(e) X5 is not Q;
(f) X6 is not L;
(g) X8 is not V;
(h) X10 is absent; or
(i) X9 and X10 are absent.

48. The biomedical device according to claim 47, wherein X4 is R, X5 is Q and X8 is V.

49. The biomedical device according to claim 40, wherein the therapeutic agent is an isolated polypeptide having at least 90% amino acid sequence identity to KAFAKLAARLYRKALARQLGVAA [SEQ ID NO: 1], wherein the polypeptide inhibits TNF-α secretion.

50. The biomedical device according to claim 40, wherein the therapeutic agent is an isolated polypeptide having at least 90% amino acid sequence identity to FAKLAARLYRKALARQLGVAA [SEQ ID NO: 2], wherein the polypeptide inhibits TNF-α secretion.

51. The method according to claim 39, wherein the hydrophilic polymer comprises acrylamide, styrene, acrylic acid and a polymerization initiator.

52. The method according to claim 39, wherein the hydrophilic polymer comprises (i) 50% acrylamide, (ii) 15% styrene, (iii) 35% acrylic acid and (iv) 2,2′-azobisisobutyronitrile 1/100 molar ratio to monomers.

53. The method according to claim 39, wherein the hydrophilic polymer comprises (i) 50% acrylamide, (ii) 15% styrene, (iii) 35% acrylic acid and (iv) 2,2′-azobisisobutyronitrile 1/200 molar ratio to monomers.

54. The method according to claim 51, wherein the acrylic acid is functionalized with a guanidyl group.

55. The method of claim 54, wherein the guanidyl group is agmatine sulfate.

56. The method of claim 54, wherein the guanidyl group is of arginine, or a derivative thereof.

57. A method for treating inflammation with a biocompatible biogel composition, the method comprising the steps:
(i) providing a biocompatible biogel composition comprising
(a) a cationic component; wherein the cationic component comprises a hydrophilic polymer having a molecular weight great than about 3000 g/mole, but less than about 10,000,000 g/mole, to which at least about 3, but no more than 1,000,000 cationic oligomers is grafted;
(b) an anionic component; and
(c) a therapeutically effective amount of a therapeutic agent;
(ii) administering the biocompatible biogel composition of step (i) to a region of interest within a subject in need thereof, wherein the region of interest contains or is adjacent to an area of inflammation; thereby reducing the inflammation.

58. The method according to claim 57, wherein the therapeutic agent is selected from the group consisting of an analgesic agent, an antimicrobial agent, a steroid agent, a chemotherapeutic agent, a biological agent, a pharmaceutical composition, a growth factor, a cell, or a polypeptide.

59. The method according to claim 57, wherein the therapeutic agent is a microparticle form.

60. The method according to claim 57, wherein the therapeutic agent is a nanoparticle form.

61. The method according to 58, wherein the biological agent is an isolated cell.

62. The method according to claim 58, wherein the biological agent is an isolated peptide, an isolated polypeptide, an isolated antibody or an isolated active portion, fragment or derivative thereof.

63. The method according to claim 58, wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid.

64. The method according to claim 58, wherein the biological agent is an isolated polypeptide having an amino acid sequence according to general formula I:
Z1-X1-X2-X3-X4 X5-X6-X7-X8-X9-X10-Z2
wherein Z1 and Z2 are independently absent or are transduction domains;
X1 is selected from the group consisting of A, KA, KKA, KKKA, and RA, or is absent;
X2 is selected from the group consisting of G, L, A, V, I, M, Y, W, and F, or is an aliphatic amino acid;
X3 is selected from the group consisting of V, L, I, A, G, Q, N, S, T, and C, or is an aliphatic amino acid;
X4 is selected from the group consisting of Q, N, H, R and K;
X5 is selected from the group consisting of Q and N;
X6 is selected from the group consisting of C, A, G, L, V, I, M, Y, W, and F or is an aliphatic amino acid;
X7 is selected from the group consisting of S, A, C, T, and G or is an aliphatic amino acid;
X8 is selected from the group consisting of V, L, I, and M;
X9 is absent or is any amino acid; and
X10 is absent or is any amino acid;
wherein at least one of the following is true:
(a) X3 is N and X7 is not G;
(b) X7 is G and X3 is not N;
(c) X2 is not L;
(d) X4 is not R;
(e) X5 is not Q;
(f) X6 is not L;
(g) X8 is not V;
(h) X10 is absent; or
(i) X9 and X10 are absent.

65. The method according to claim 64, wherein X4 is R, X5 is Q and X8 is V.

66. The method according to claim 57, wherein the therapeutic agent is an isolated polypeptide having at least 90% amino acid sequence identity to KAFAKLAARLYRKALARQLGVAA [SEQ ID NO: 1], wherein the polypeptide inhibits TNF-α secretion.

67. The method according to claim 57, wherein the therapeutic agent is an isolated polypeptide having at least 90% amino acid sequence identity to FAKLAARLYRKALARQLGVAA [SEQ ID NO: 2], wherein the polypeptide inhibits TNF-α secretion.

68. The method according to claim 57, wherein the hydrophilic polymer comprises acrylamide, styrene, acrylic acid and a polymerization initiator.

69. The method according to claim 57, wherein the hydrophilic polymer comprises (i) 50% acrylamide, (ii) 15% styrene, (iii) 35% acrylic acid and (iv) 2,2′-azobisisobutyronitrile 1/100 molar ratio to monomers.

70. The method according to claim 57, wherein the hydrophilic polymer comprises (i) 50% acrylamide, (ii) 15% styrene, (iii) 35% acrylic acid and (iv) 2,2′-azobisisobutyronitrile 1/200 molar ratio to monomers.

71. The method according to claim 68, wherein the acrylic acid is functionalized with a guanidyl group.

72. The method according to claim 71, wherein the guanidyl group is agmatine sulfate.

73. The method according to claim 71, wherein the guanidyl group is of arginine, or a derivative thereof.

74. The method according to claim 57, whereby the inflammatory disorder is selected from the group consisting of hyperplastic scarring, keloids, rheumatoid arthritis, chronic obstructive pulmonary disease, atherosclerosis, intimal hyperplasia, Crohn's disease, inflammatory bowel disease, osteoarthritis, Lupus, tendonitis, psoriasis, gliosis, inflammation, type II diabetes mellitus, type I diabetes mellitus, Alzheimer's disease, and an adhesion.

75. The method according to claim 57, wherein the inflammatory disorder comprises glial scarring.

76. A tissue filler to fill a tissue void, comprising
(a) a gel-like system comprising
(i) a cationic component, wherein the cationic component comprises a hydrophilic polymer having a molecular weight great than about 3000 g/mole, but less than about 10,000,000 g/mole, to which at least about 3, but no more than 1,000,000 cationic oligomers is grafted; and
(ii) an anionic component;
(b) and optionally a therapeutically effective amount of a therapeutic agent.
